(19) United States
(12) Patent Application Publication
(10) Pub. No.: US 2006/0188878 A1
(43) Pub. Date: Aug. 24, 2006

(54) Methods to predict ederma as a side effect of drug treatment

(76) Inventors: Marlene Michelle Dressman, Germantown, MD (US); Sridhar Kudaravalli, Chicago, IL (US); Rachel Helene Malinowski, Pittsburgh, PA (US); Lee Anne McLean, Medway, MA (US); Mihael Hristos Polymeropoulos, Potomac, MD (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/530,391

(22) PCT Filed: Oct. 14, 2003
(86) PCT No.: PCT/EP03/11377

§ 371 Date: Feb. 2, 2006

Related U.S. Application Data
(60) Provisional application No. 60/418,556, filed on Oct. 15, 2002.

(51) Int. Cl. C12Q 1/68

(52) U.S. Cl. 435/6

(57) ABSTRACT

This invention provides methods to predict the likelihood of occurrence of the side effect of edema in patients treated with a drug including, but not limited to, a TKI, such as Imatinib or GLEEVEC™/GLIVEC®. The methods employed use gene expression profile comparisons and the determination of specific SNPs and in the IL-1β gene. Methods of treatment of edema and kits for the performance of the above assays are also provided.

### BACKGROUND OF THE INVENTION

1. Field of the Invention

This invention relates to methods to predict the likelihood of occurrence of edema in a patient treated with a drug, including but not limited to a tyrosine kinase inhibitor (TKI) drug. In particular, this invention relates to the use of several forms of genomic analysis to predict the occurrence of edema as a side effect in patients treated with drugs, including TKI drugs, such as Imatinib, especially the mesylate salt therof (GLEEVEC™/GLIVEC®; also known as STI571, Novartis Pharmaceuticals, East Hanover, N.J., USA). The type of genomic analyses includes gene expression profiling and the detection of single nucleotide polymorphisms (SNPs).

2. Description of Related Art

Edema is defined as an increase in the extravascular or interstitial component of the extracellular fluid volume. Edema may come in many forms, thus fluid may accumulate in the peritoneal or pleural cavities or may be generalized, as in anasarca. The location and distribution of edema is determined by its etiology and mechanism. Edema is often recognized by puffiness in the face, which is most apparent in the periorbital areas and by the persistence of an indentation of the skin following pressure, this is known as pitting.

In general the plasma volume and the interstitial volume comprise the “extracellular space” which holds one-third of the total body water. The forces that regulate the disposition of fluid between the two components of the extracellular compartment are called Starling forces. Generally, the hydrostatic pressure within the vascular system and the colloid oncotic pressure in the interstitial fluid tend to promote movement of fluid from the vascular to the extravascular space. In contrast the colloid oncotic pressure contributed by the plasma proteins and the hydrostatic pressure within the interstitial fluid referred to as tissue tension, promote the movement of fluid into the vascular compartment. As a consequence of the forces there is a constant exchange of fluids and diffusible solutes Edema may result from any disturbance in these forces, such as an increase in capillary pressure or permeability.

Of particular interest here, are the various forms of edema which are caused, as a side effect, of the therapeutic administration of drugs. The mechanisms by which these drugs produce edema in a patient are not generally known. The edema produced in response to most drugs is fairly mild, such as barely noticeable periorbital edema, however it is possible for a drug to produce life-threatening forms of edema, such as pulmonary edema of cerebral edema.

Imatinib is an inhibitor of the tyrosine kinase activity of several proteins that play a causative or very significant role in the development of cancers of several types, however, its use can in some cases cause the development of edema. See Druker et al., Nature Med., Vol. 2, pp. 561-566 (1996).

Background on Leukemia

The various forms of leukemia comprise a variety of related disorders with similar underlying pathology. The basic pathology is a dysregulation of normal hematopoiesis. This process requires tightly regulated proliferation and differentiation of pluripotent hematopoietic stem cells that become mature peripheral blood cells. In all types of leukemia, the malignant event or events occur somewhere in the hematopoietic progression and results, by different mechanisms, in giving rise to progeny that fail to differentiate normally and instead continue to proliferate in an uncontrolled fashion. Leukemias are divided into acute and chronic types and into myeloid and lymphocytic type depending on the cell line affected and the rate of progression.

Chronic myelogenous leukemia (CML) is also called chronic myeloid leukemia, chronic myelocytic leukemia or chronic granulocyte leukemia. CML is a disease characterized by overproduction of cells of the granulocytic, especially the neutrophilic series and occasionally the monocytic series, leading to marked splenomegaly and very high white blood cell counts. Basophilia and thrombocytosis are common. A characteristic cytogenetic abnormality, the Philadelphia (Ph′) chromosome, is present in the bone marrow cells in more than 95% of cases. The presence of this altered chromosome is both the key to understanding the molecular pathogenesis of this type of leukemia and a major index to assess clinical improvement in patients. See Sawyer, N. Engl. J. Med., Vol. 340, pp. 1330-1340 (1999).

The most striking pathological feature in CML is the presence of the Ph′ chromosome in the bone marrow cells of more than 90% of patients with typical CML. The Ph′ chromosome results from a balanced translocation of material between the long arms of chromosomes 9 and 22. As more chromosomal material is lost from chromosome 22 than is gained from chromosome 9, the Ph′ chromosome is a shortened chromosome 22 containing approximately 60% of its normal complement of DNA. The break, which occurs at band q34 of the long arm of chromosome 9, allows translocation of the cellular oncogene C-ABL to a position on chromosome 22 called the breakpoint cluster region (BCR). The breakpoint in the BCR varies from patient to patient but is identical in all cells of any one patient. C-ABL is a homologue of V-ABL, the Abelson virus that causes leukemia in mice. The apposition of these two genetic sequences produces a new hybrid gene (BCR/ABL), which codes for a novel protein of molecular weight 210,000 kd (P210). The P210 protein, a tyrosine kinase, may play a role in triggering the uncontrolled proliferation of CML cells. The Ph′ chromosome occurs in erythroid, myeloid, monocytic and megakaryocytic cells, less commonly in B lymphocytes, rarely in T lymphocytes, but not in marrow fibroblasts.

In the past, the prognosis for CML was poor with the mean survival in Ph-positive (Ph⁺) CML being 3-4 years. Treatment with interferon and aggressive chemotherapy or allogeneic bone marrow transplant has improved this somewhat but the greatest improvement in the treatment of CML patients has been the introduction of Imatinib. See Druker et al., N. Engl. J. Med., Vol. 344, pp. 1031-1037 (2001); and Druker et al., N. Engl. J. Med., Vol. 344, pp. 1038-1056 (2001); and also see Cecil Textbook of Medicine, 21^{st} Edition, Goldman and Bennett, Eds., W. B. Saunders, Chapter 176 (2000).

In CML, chromosomes 9 and 22 are truncated in the formulation of the ⁺ (9;22) reciprocal translocation that characterizes CML cells and two fusion genes are generated: BCR-ABL on the derivative 22q-chromosome, the Ph′ chromosome and ABL-BCR on chromosome 9q⁺. The BCR-ABL gene encodes a 210-kd protein with deregulated tyrosine kinase activity. This protein plays a pathogenetic role in CML. See Daley et al., Science, Vol. 247, pp. 824-830 (1990). Imatinib specifically inhibits the activity of this protein and other tyrosine kinases. Imatinib has shown remarkable efficacy in treating patients with CML and in treating patients in blast crisis of CML or ALL (acute lymphoblastic leukemia) with the Ph′ chromosome. See Druker (2001), supra.

In addition, the ability of Imatinib to inhibit another tyrosine kinase that is a growth factor receptor terminal, i.e., c-Kit, allows Imatinib to be an effective treatment for a completely unrelated form of cancer, gastrointestinal stromal tumors. See Brief Report, Joensuu et al., N. Engl. J. Med., Vol. 344, No. 14, pp. 1052-1056 (2001).

Imatinib has been shown to be highly effective in patients having a variety of disorders characterized by the uncontrolled activity of a tyrosine kinase. This includes Ph⁺ leukemia. In one study of the effects of Imatinib on CML, of 54 patients who were treated with 300 mg or more, 53 had complete hematologic responses, and cytogenic responses occurred in 29 including 17 (31% of the 54 patients who received the dose) with major responses, i.e., 0-35% of cells in metaphase positive for the Ph′ chromosome; 7 of these patients had complete cytogenetic remission. See Druker et al. (2001), supra.

Imatinib was developed as a specific inhibitor of the BCR-ABL tyrosine kinase and has been demonstrated to be highly effective in the treatment of CML patients. While generally well-tolerated, edema has been cited as one of the most commonly experienced side effects of Imatinib treatment. See Kantarjian et al., N. Engl. J. Med., Vol. 346, pp. 645-652 (2002); Druker et al. (2001), supra; Cohen et al., Clin. Cancer Res., Vol. 8, pp. 935-942 (2002); and Ebnöether et al., Lancet, Vol. 359, pp. 1751-1752 (2002).

Reports of clinical trials for patients with CML in chronic phase indicate edema/fluid retention as one of the most common adverse events associated with Imatinib treatment, occurring in 39-60% of patients (all grades). See Kantarjian et al. (2002), supra; Druker et al. (2001), supra; and Cohen et al. (2002), supra. Most of these cases were of minor to moderate severity and were primarily superficial, e.g., periorbital edema and peripheral edema of the lower extremities. However, in 1-2% of patients, more serious forms of fluid retention were seen, including pulmonary edema, pleural and pericardial effusions. See Cohen et al. (2002), supra. Furthermore, there was a recent report of 2 cases of cerebral edema, one of which was fatal, in CML patients treated with Imatinib. See, Ebnöether et al. (2002), supra.

While the majority of reported cases of edema are of mild to moderate severity, consisting primarily of periorbital edema and peripheral edema of the lower extremities. See Cohen et al. (2002), supra. As discussed above, there have been rare occurrences of more severe forms of edema, including 2 recently reported cases of cerebral edema in CML patients treated with Imatinib. See Ebnöether et al. (2002), supra. This potential for more serious cases of edemas makes it vital that methods for predicting the likelihood that a patient in need of treatment with a TKI will develop edema as a side effect to that treatment be developed. Prior to this invention there was no way to predict this potentially serious side effect of this important class of drugs.

### SUMMARY OF THE INVENTION

The present invention solves the problem mentioned above by providing a number of methods and kits by which it is possible to predict the likelihood that a given patient will develop edema as a side effect to treatment with a drug including, but not limited to, TKI drugs including, but not limited to, Imatinib or GLEEVEC™/GLIVEC®. These methods and kits rely on gene expression profiling and analysis of SNPs in several genes.

Thus, one aspect of this invention is a method to predict which patients will be more likely to develop edema when treated with a drug including, but not limited to, a TKI drug comprising: a) determining RNA expression levels in a biological sample for a plurality of the 13 reporter/predictor genes shown in Table 2; b) comparing patients gene expression profile to the mean No Edema expression profiles shown in Table 3; c) determining the similarity between the two gene expression profiles resulting from the comparison in (b); and d) determining the likelihood that the patient will develop edema when treated with a drug by means of the degree of similarity determined in (c). In more preferred embodiments the methods entail using the method above wherein the said similarity determined in (c) is the mathematical correlation coefficient obtained by comparing the said two gene expression profiles. In most preferred embodiments of this invention, the said correlation coefficient determined in (c) is the Pearson Correlation Coefficient (PCC).

In other preferred embodiments, this invention provides a method to predict which patients will be more likely to develop edema when treated with a drug including, but not limited to, a TKI drug comprising: a) determining RNA expression levels in a biological sample for a plurality of the 13 reporter/predictor genes shown in Table 2; b) comparing patients gene expression profile to the mean No Edema expression profiles shown in Table 3; c) determining the Pearson Correlation Coefficient (PCC) between the two gene expression profiles resulting from the comparison in (b); d) determining that the patient will be more likely to develop edema than not, when treated with a drug, if the PCC is <0.37; and e) determining that the patient will be more likely not to develop edema than to develop it if the PCC is ≧0.37.

In another embodiment, where it is necessary to predict with high sensitivity which patients will be more likely to develop edema when treated with a drug including, but not limited to, a TKI drug, such that no more than 15% of Edema cases will be misclassified as having No Edema, this invention provides a method, comprising: a) determining RNA expression levels in a biological sample for a plurality of the 13 reporter/predictor genes shown in Table 2; b) comparing patients gene expression profile to the mean No Edema expression profiles shown in Table 3; c) determining the PCC between the two gene expression profiles resulting from the comparison in (b); d) determining that the patient will be more likely to develop edema than not, when treated with a drug, if the PCC is negative and <0.78; and e) determining that the patient will be more likely not to develop edema than to develop it if the negative PCC is ≧0.78.

In preferred embodiments of the invention the biological sample comprises a blood or a tissue sample. Suitable blood or tissue samples include whole blood, serum, semen, saliva, tears, urine, fecal material, sweat, buccal smears, skin and biopsies of specific organ tissues, such as muscle or nerve tissue and hair.

According to other embodiments, the invention provides methods, wherein the RNA expression level of 7 or 8, more preferably of 9 or 10, and most preferably of 11 or 12 of the 13 reporter/predictor genes is determined. Other embodiments of the invention provide methods, wherein the RNA levels of all the 13 reporter/predictor genes in Table 2 are determined.

In another aspect, this invention provides a method to predict which female patients will be more likely to develop edema when treated with a drug including, but not limited to, a TKI drug comprising: a) determining for the two copies of the IL-1β gene present in the patient, the identity of the nucleotide pairs at the polymorphic site at position −511 base pairs upstream (at position 1423 of sequence X04500) from the transcriptional start site; b) determining that the patient will be likely to develop edema if both nucleotide pairs at this site are GC; and c) determining that the patient will not be likely to develop edema if at least one nucleotide pair at this site is AT.

In another aspect, this invention provides a method to predict which female patients will be more likely to develop edema when treated with a drug including, but not limited to, a TKI drug, comprising: a) determining for the two copies of the IL-1β gene present in the patient, the identity of the nucleotide pairs at the polymorphic site at position −31 base pairs upstream (at position 1903 of sequence X04500) from the transcriptional start site; b) determining that the patient will be likely to develop edema if both nucleotide pairs at this site are AT; and c) determining that the patient will not be likely to develop edema if at least one nucleotide pair at this site is GC.

Other aspects of the invention provide for methods to predict which female patient will be more likely to develop edema when treated with a drug, comprising step a) determination of the level of transcription of the IL-1β gene and/or of the level of the protein expressed by the IL-1β gene in a biological sample; and b) determining that the patient would be likely to develop edema when treated with a drug if the level is above a threshold level.

In addition, this invention provides the above methods wherein the drug is the TKI Imatinib or GLEEVEC™/GLIVEC®.

Furthermore, this invention provides a method to predict which patients will be more likely to develop edema when treated with a drug comprising: a) determining the pattern of protein expression in a biological sample for two or more of the protein products of the 13 predictor genes shown in Table 2; b) comparing the pattern of protein expression with the pattern expected for the Edema and the No Edema expression profile shown in Table 3; c) determining that if the pattern is more similar to the No Edema pattern that the patient will not be likely to develop edema when treated with a drug; and d) determining that if the pattern is more similar to the Edema pattern that the patient will be likely to develop edema when treated with a drug. In this method the drug may be any TKI including but not limited to Imatinib or GLEEVEC™/GLIVEC®.

In other preferred embodiments of the methods according to the invention the biological sample comprises blood drawn from a patient. Alternatively, the level of transcription or the level of protein expression is determined in other biological samples such as serum or tissue samples obtainable from the patient including semen, saliva, tears, urine, fecal material, sweat, buccal smears, skin and biopsies of specific organ tissues, such as muscle or nerve tissue and hair.

Other embodiments of the invention provide methods, wherein the protein expression of a plurality of the 13 predictor genes shown in Table 2 is determined. Preferably, the protein expression of 7 or 8, more preferably of 9 or 10, and most preferably of 11 or 12 of the 13 predictor genes is determined. In another most preferred embodiment a method is provided, wherein the protein expression of all the 13 predictor genes shown in Table 2 is determined.

In other preferred aspects this invention provides a method to design clinical trials for the testing of drugs comprising: a) determining by the use of either the expression profiling or the genotyping methods described above the likelihood that a particular patient will develop edema when exposed to the test drug; and b) assigning that patient to the appropriate classification in the clinical study based on the results of the determination in (a).

In addition this invention provides a method to treat a patient with a drug comprising: a) determining by the use of either the expression profiling or the genotyping methods described above the likelihood that the particular patient will develop edema when exposed to the intended drug; and b) modifying the intended drug therapy for that patient in a safe and appropriate manner based on the results of the determination in (a).

In some preferred embodiments this invention provides a method of treating a subject having, or at risk of having, edema comprising administering to the subject a therapeutically effective amount of an isolated nucleic acid molecule comprising an antisense nucleotide sequence derived from the IL-1β gene, which has the ability to change the transcription/translation of the IL-1β gene.

In some other preferred embodiments this invention provides a method of treating a subject having, or at risk of having, edema comprising administering to the subject a therapeutically effective amount of an antagonist that inhibits/activates the protein encoded by the IL-1β gene. In using this method the antagonist may be any antibody, antibody derivatives, or antibody fragments specific for the protein, including but not limited to a monoclonal antibody and/or a monoclonal antibody conjugated to a toxic reagent.

In some other preferred embodiments this invention provides a method of treating a subject having, or at risk of having, edema comprising administering to the subject a therapeutically effective amount of a nucleotide sequence encoding a ribozyme, which has the ability to change the transcription/translation of the IL-1β gene.

In some preferred embodiments this invention provides a method of treating a subject having, or at risk of having, edema comprising administering to the subject a therapeutically effective amount of a double-stranded RNA corresponding to the IL-1β gene, which has the ability to change the transcription/translation of the IL-1β gene.

In most preferred embodiments this invention provides methods as described above, wherein the transcription/translation of the IL-1β gene is decreased. In other most preferred embodiments the transcription/translation of the IL-1β gene is increased.

Another aspect of the invention provides a kit for predicting which patient will be more likely to develop edema when treated with a drug comprising a means for determining the pattern of protein expression corresponding to two or more of the 13 predictor genes shown in Table 2. According to a preferred embodiment of the invention the means is able to determine the pattern of protein expression corresponding to a plurality of the 13 predictor genes. Preferably, the protein expression of 7 or 8, more preferably of 9 or 10, and most preferably of 11 or 12 of the 13 predictor genes is determined. In another most preferred embodiment a kit is provided, wherein the means is able to determine the protein expression of all the 13 predictor genes shown in Table 2.

Another aspect of the invention provides a kit for predicting which patient will be more likely to develop edema when treated with a drug comprising a means for determining the level of the protein expressed by the IL-1β gene.

In preferred embodiments of the invention the means for determining the pattern of protein expression comprises antibodies, antibody derivatives, or antibody fragments. A suitable method to determine the protein expression includes Western blotting utilizing a labeled antibody.

A most preferred embodiment of the invention provides a kit for predicting which patient will be more likely to develop edema when treated with a drug comprising: (a) a means for determining the pattern of protein expression corresponding to the two or more of the 13 predictor genes shown in Table 2; (b) a container suitable for containing the said means and the biological sample of the patient comprising the proteins, wherein the means can form complexes with the proteins; (c) a means to detect the complexes of (b); and optionally (d) instructions for use and interpretation of the kit results.

In other embodiments this invention provides a kit for determining the protein expression pattern for the 13 predictor genes shown in Table 2 comprising: a) a container comprising or containing all the reagent necessary to determine the protein expression pattern; and b) a label describing how to perform and interpret the analysis.

Another aspect of the invention provides a kit for predicting which patient will be more likely to develop edema when treated with a drug comprising: (a) a means for determining the level of the protein expressed by the IL-1β gene; (b) a container suitable for containing the said means and the biological sample of the patient comprising the protein, wherein the means can form complexes with the protein; (c) a means to detect the complexes of (b); and optionally (d) instructions for use and interpretation of the kit results.

In preferred embodiments of the invention the level of the protein expressed by the IL-1β gene is determined in blood or in serum.

Another aspect of the invention provides a kit for predicting which patient will be more likely to develop edema when treated with a drug comprising a means for determining the level of transcription of two or more of the 13 predictor genes shown in Table 2. According to a preferred embodiment of the invention the means is able to determine the level of transcription of a plurality of the 13 predictor genes. Preferably, the level of transcription of 7 or 8, more preferably of 9 or 10, and most preferably of 11 or 12 of the 13 predictor genes is determined. In another most preferred embodiment a kit is provided, wherein the means is able to determine the level of transcription of all the 13 predictor genes shown in Table 2.

Another aspect of the invention provides a kit for predicting which patient will be more likely to develop edema when treated with a drug comprising a means for determining the level of transcription of the IL-1β gene.

In preferred embodiments of the invention the means for determining the level of transcription comprise oligonucleotides or polynucleotides able to bind to the transcription products of said genes as described above; most preferably the oligonucleotides or polynucleotides are able to bind mRNA or cDNA corresponding to the predictor genes or the IL-1β gene. Suitable methods to determine the level of transcription include Northern blot analysis, reverse transcriptase PCR, real-time PCR, RNAse protection, and microarray.

In another preferred embodiments of the invention the kits as described above further comprise means for obtaining a biological sample of the patient. Preferably biological samples taken from a patient comprise a blood or a tissue sample. Suitable blood or tissue samples include whole blood, serum, semen, saliva, tears, urine, fecal material, sweat, buccal smears, skin and biopsies of specific organ tissues, such as muscle or nerve tissue and hair. In a preferred embodiment a kit comprises further a container suitable for containing the means for detecting the proteins or the means for measuring the level of transcription and the biological sample of the patient, and optionally further comprises instructions for use and interpretation of the kit results.

Another most preferred embodiment of the invention provides a kit for predicting which patient will be more likely to develop edema when treated with a drug comprising: (a) a number of oligonucleotides or polynucleotides able to bind to the transcription products of the two or more of the 13 predictor genes shown in Table 2; (b) a container suitable for containing the oligonucleotides or polynucleotides and the biological sample of the patient comprising the transcription products wherein the oligonucleotides or polynucleotide can bind to the transcription products; (c) means to detect the binding of (b); and optionally (d) instructions for use and interpretation of the kit results.

In alternate embodiments this invention provides a kit for determining the expression pattern of the 13 predictor genes shown in Table 2 comprising: a) a container comprising or containing the necessary gene chip along with the needed reagents to develop it; and b) instructions for the preparation, reading and interpretation of the resulting gene expression patter.

Another embodiment of the invention provides a kit for predicting which patient will be more likely to develop edema when treated with a drug comprising: (a) oligonucleotides or polynucleotides able to bind to the transcription products of the IL-1β gene; (b) a container suitable for containing the oligonucleotides or polynucleotides and the biological sample of the patient comprising the transcription products wherein the oligonucleotides or polynucleotide can bind to the transcription products; (c) means to detect the binding of (b); and optionally (d) instructions for use and interpretation of the kit results.

Preferably the drug according to the above described aspects or embodiments of the invention may be any TKI including but not limited to Imatinib or GLEEVEC™/GLIVEC®.

In addition, the invention provides kits for the identification of a polymorphism pattern at the IL-1β gene of a patient, said kits comprising a means for determining the genetic polymorphism pattern at the IL-1β gene at position 1423 of sequence X04500 and/or at position 1903 of sequence X04500. The kit may further comprise a means for obtaining a biological sample of the patient, including blood or tissue samples such as whole blood, serum, semen, saliva, tears, urine, fecal material, sweat, buccal smears, skin and biopsies of specific organ tissues, such as muscle or nerve tissue and hair. Preferably such means comprises a DNA sample collecting means.

According to preferred embodiments of the invention, the means for determining a genetic polymorphism pattern at the specific polymorphic site comprises at least one gene specific genotyping oligonucleotide. Most preferably the kit comprises two gene specific genotyping oligonucleotides. Alternatively, the kit comprises four gene specific genotyping oligonucleotides. In an even more preferred embodiment the kit comprises at least one gene specific genotyping primer composition comprising at least one gene specific genotyping oligonucleotide. Preferably such gene specific genotyping primer composition comprises at least two sets of allele specific primer pairs, which are optionally packaged in separate containers.

In addition, this invention provides a kit for determining the identity of the nucleotide pair at the −511 position of the IL-1β gene (at position 1423 of sequence X04500) from the transcriptional start site for the two copies of the IL-1β gene present in the patient; comprising: a) a container comprising or containing at least one reagent specific for detecting the nature of the nucleotide pair at the at the −511 position of the IL-1β gene (at position 1423 of sequence X04500) from the transcriptional start site for the two copies of the IL-1β gene present in the patient; and b) instructions for interpreting the results based on the nature of the said nucleotide pair.

In addition, this invention provides a kit for determining the identity of the nucleotide pair at the polymorphic site at position −31 base pairs upstream (at position 1903 of sequence X04500) from the transcriptional start site; comprising: a) a container comprising or containing at least one reagent specific for detecting the nature of the nucleotide pairs at the polymorphic site at position −31 base pairs upstream (at position 1903 of sequence X04500) from the transcriptional start site; and b) instructions for interpreting the results based on the nature of the said nucleotide pair.

Furthermore, other embodiments of the inventions provide that any one of the above described kits are used in determination step (a) of methods provided by the invention including the methods to predict which patient including which female patient will be more likely to develop edema when treated with a drug such as a TKI including but not limited to Imatinib or GLEEVEC™/GLIVEC®.

### BRIEF DESCRIPTION OF THE FIGURES

FIG. 1: Cluster analysis of the optimal 13 genes used to predict edema for the 88-sample “predictor” set. The degrees of shading represent relative levels of expression, with the darkest shading representing low expression and the intermediate shading representing high expression. Samples are ordered according to correlation of gene expression with the mean No Edema expression profile and clustering of genes was performed using the Pearson similarity method in GENESPRING®. PCCs for each sample are plotted in the middle panel, with highest correlation at the top. The right panel represents the actual edema status, with solid (dark) indicating Edema and white representing patients with No Edema. The lines indicate threshold values for optimum accuracy (0.37; solid line) and optimal sensitivity (0.78; dashed line).

FIG. 2: Cluster analysis for the 17-sample “test” set used to validate the edema predictor genes. The degrees of shading represent relative levels of expression, with the darkest shading representing low expression and intermediate shading representing high expression. Samples are ordered according to correlation of gene expression with the mean No Edema expression profile (calculated from the 88-sample “predictor” set) and clustering of genes was performed using the Pearson similarity method in GENESPRING®. PCCs for each sample are plotted in the middle panel, with highest correlation at the top. The right panel represents the actual edema status, with solid (dark) indicating Edema and white representing patients with No Edema. The dashed line indicates the threshold at optimal sensitivity (0.78), as determined using the 88-sample “predictor” set.

FIG. 3: Association of IL-1β genotype with edema and angioedema. CC genotype refers the presence of GC base pair on both copies of the IL-1β gene at the polymorphic site −511 (at position 1423 of sequence X04500 gene). Non-CC genotype refers to the presence of AT base pair on one or both copies at the polymorphic site −511 of the IL-1β gene (at position 1423 of sequence X04500 gene), i.e. it refers to the C→T base transition at the −511 base pairs upstream from the transcriptional start site.

FIG. 4: Association of the −511 IL-1β polymorphism with edema stratified by sex.

### DETAILED DESCRIPTION OF INVENTION

The present invention provides several different methods to predict the likelihood of occurrence of the side effect of edema in patients who are treated with drugs including, but not limited to, a drug that is an inhibitor of the tyrosine kinase activity of several proteins, i.e., a tyrosine kinase inhibitor (TKI) drug, this includes, but is not limited to, Imatinib, Imatinib mesylate or GLEEVEC™/GLIVEC® also known as STI571, Novartis Pharmaceuticals, East Hanover, N.J., USA.

In one embodiment, a patient in need of treatment with a drug such as a TKI would have blood drawn for a determination of the RNA expression profile comprising a plurality of the 13 genes shown in Table 2. Alternatively the RNA expression levels may be determined in other tissue samples including semen, saliva, tears, urine, fecal material, sweat, buccal smears, skin and biopsies of specific organ tissues, such as muscle or nerve tissue and hair. In one embodiment the measured RNA expression levels for this group of genes would be compared to the mean Edema expression levels or the mean No Edema expression levels for the same 13 predictor genes as shown in Table 3 and the degree of similarity determined.

In a preferred embodiment, the measured RNA expression levels from the patient for this group of 13 predictor genes would be compared to the mean No Edema expression levels for the same genes as shown in Table 3 and the degree of similarity determined.

The degree of similarity can be determined by any mathematical procedure that produces a result whose value is a known function of the similarity between the two groups of numbers, i.e., the measured mRNA expression values from the patients blood for a plurality of the 13 predictor genes and the mean No Edema expression values, or the mean Edema expression values, shown in Table 3.

In a preferred embodiment, the degree of similarity is determined by determining a mathematical correlation coefficient, including but not limited to the Pearson Correlation Coefficient (PCC), between the patients measured RNA expression levels and the mean No Edema RNA expression levels of a plurality of the 13 genes shown in Table 3.

In a most preferred embodiment, the correlation coefficient is the Pearson Correlation Coefficient (PCC) and all 13 predictor genes are used to make the comparison most accurate. The value of the PCC so determined, or any other correlation coefficient or similarity index, can then be used to predict the likelihood of the occurrence of edema if the patient is then treated with an edema producing drug, including but not limited to a TKI drug including but not limited to Imatinib or Imatinib mesylate or GLEEVEC™/GLIVEC®.

In a preferred embodiment, the degree of similarity between the patients measured RNA expression profile and mean Edema or the mean No Edema expression profile (from Table 3) can then be used to predict whether the patient is likely to develop edema when treated with a TKI drug or not. Thus to state it simply, if the patients' measured RNA expression profile for all or most of the 13 genes shown in Table 2 is more similar to the mean expression profile for the subjects who did not develop edema (mean No Edema expression profile) then the likelihood that this patient will develop edema when treated with a TKI drug is small. If the patients' measured RNA expression profile for all or most of the 13 genes shown in Table 3 is more similar to the mean expression profile of the subjects who did develop edema of any kind (mean Edema expression profile) when treated with a TKI drug, then that patient is more likely to develop edema when treated with a drug, such as a TKI.

In a preferred embodiment, this degree of similarity is determined by calculation of the PCC between the measured patients gene expression profile for the 13 genes in Table 2 and mean expression profile from the No Edema patients (Table 3).

The value of the PCC is directly related to the probability that the patient will suffer the same side effect of Edema or No Edema as the Table 3 expression profile to which it is compared. That is to say, the higher the patients' PCC as compared to the mean No Edema expression profile, the higher the likelihood that the patient will not develop edema in response to a TKI drug. On the other hand, the higher the patients' PCC is as compared to the mean Edema expression profile, then the higher the likelihood that the patient will develop edema when treated with a drug, such as a TKI.

Thus, in a given case the value of the PCC, can be used to determine probabilities for the outcome, that is to say the development of edema or not if the patient is treated with a drug, such as a TKI including, but not limited to, Imatinib or GLEEVEC™/GLIVEC®. Those of skill in the art will understand that the clinical circumstance for each patient will dictate the value of the PCC to be used as a cutoff or to help make clinical decisions with regard to a specific patient. For example, in one embodiment, it is desirable to determine with optimal accuracy the number of a group of patients who will and who will not develop edema. This means to minimize both false positives (No Edema misclassified as Edema) and at the same time to minimize false negatives (Edema misclassified as No Edema).

This degree of accuracy can be had by setting the PCC at 0.37. To use this threshold, a patient whose gene expression profile when compared with the mean No Edema expression profile achieves a PCC of ≧0.37 would be classified as the No Edema group, while a patient whose expression profile was <0.37 would be classified as the Edema group.

In a further preferred embodiment, the PCC can be set to produce optional sensitivity. That is, to make the smallest possible number of false negatives (Edema misclassified as No Edema). Such an optimal sensitivity setting would be indicated in situations where the occurrence of edema would be a serious or life-threatening event for the patient. In this embodiment, the threshold is determined by setting the PCC to 0.78. In this case, the patient is 7.20 (95% confidence interval (CI): 2.42-21.44) times more likely to develop edema if their expression profile is negatively correlated with the mean No Edema profile with a PCC of <0.78. As is shown in the example, one of skill in the art can choose a degree of similarity or correlation coefficient, including but not limited to the PCC, that will either maximize sensitivity or maximize specificity or produce any desired ratio of false positives or false negatives. One of skill in the art can easily adjust their choice of PCC to the clinical situation to provide maximum benefit and safety to the patient.

In another embodiment, this invention provides other methods to predict which patients are likely to experience edema when treated with a drug, such as a TKI. These methods involve drawing the patients blood and determining the presence or absence of certain polymorphisms in the IL-1β gene. Alternatively, other tissue samples may be obtained from a patient and used for determining the presence or absence of the IL-1β gene polymorphisms. Such tissue samples include semen, saliva, tears, urine, fecal material, sweat, buccal smears, skin and biopsies of specific organ tissues, such as muscle or nerve tissue and hair.

Specifically women patients with a CC genotype for the −511 polymorphism of the IL-1β gene are 13.0 times more likely to experience edema when treated with a TKI drug then women with a non-CC genotype (95% CI: 2.07-81.48). This polymorphism has no predictive values in male patients.

Therefore, a female patient who is about to receive a drug, such as a TKI, would have blood drawn and a determination made for the two copies of the IL-1β gene present in the patient the identity of the nucleotide pairs at the polymorphic site −511 C→T (at position 1423 of sequence X04500) of the IL-1β gene. If both nucleotides are found to be GC, then it would be predicted that the woman will develop edema when treated with a TKI drug. If both pairs are AT or one is AT and one is GC, then it would be predicted that TKI treatment would not cause the side effect of edema.

In another embodiment of this invention, a female patient who is about to receive a drug, such as a TKI, would have blood drawn and a determination made for the two copies of the IL-1β gene present in the patient the identity of the nucleotide pair at the polymorphic site −31 base pairs upstream from the transcriptional start site (at position 1903 of sequence X04500) and a determination made that the patient will be likely to develop edema with drug treatment if both nucleotide pairs at this site are AT and a determination made that the patient will not be likely to develop edema with drug treatment if at least one nucleotide pair at this site is GC.

In a still further embodiment, this invention provides kits for determining the nucleotide pairs at the polymorphic sites of interest in the IL-1β gene in a patient (both the −511 and the −31 sites), comprising: a) a container comprising or containing at least one reagent specific for detecting the nature of the nucleotide pairs at the polymorphic sites in the IL-1β gene; and b) instructions for interpretation of the results based on the nature of the said nucleotide pairs.

In a further embodiment, this invention provides a kit for determining the expression pattern of the 13 predictor genes shown in Table 2 comprising: a) a container comprising or containing the necessary gene chip along with the needed reagents to develop it; and b) instructions for the preparation, reading and interpretation of the resulting gene expression pattern.

### EXAMPLE 1

Method

The RNA Expression Profile Correlation Method

Clinical samples were obtained from patients enrolled in a multi-national Phase III clinical trial (IRIS: International Randomized Study of Interferon-α vs. Imatinib) with newly diagnosed Ph⁺ CML in chronic phase (CML-CP). Blood for RNA extraction was collected from more than 200 patients from multiple centers in the United States. Each of these patients signed a written pharmacogenetics informed consent form that was approved by local ethics committees. A total of 115 samples were collected at baseline, prior to drug treatment, from patients that were randomized to the Imatinib treatment arm. Ten of these samples were excluded from analysis due to early withdrawal of the patient from the study or because of very poor quality of the processed RNA. Of the remaining 105 samples, 88 samples were used as a “predictor” set to identify genes that could predict whether a patient would develop edema following Imatinib treatment, and 17 samples were used as a “test” set to validate the predictor genes.

Clinical data for adverse events was evaluated following a minimum of 6 months of treatment with Imatinib. A patient was identified as having edema if they experienced at least one occurrence (regardless of grade) of edema as classified using the High Level Term (HLT) of the Medical Dictionary for Regulatory Activities Terminology (MedDRA). Of the patients evaluated in this pharmacogenomics study, 43% (45 of 105) were classified as having experienced at least one episode of edema following treatment with Imatinib (Edema group), with the majority of these cases being periorbital edema (31%) and peripheral edema (19%). With the exception of a single incidence of a grade 3 periorbital edema (which was not attributed to the study medication), all cases of edema for patients evaluated in this study were of mild to moderate severity. The breakdown of edema cases was as follows: the 88-sample “predictor” set had 37 Edema and 51 No Edema; the 17-sample “test” set had 8 Edema and 9 No Edema.

RNA Expression Profiling

RNA expression data was generated from each blood sample using high-density oligonucleotide microarrays (HG U95Av2, Affymetrix, Santa Clara, Calif., USA) that represent more than 12,000 known human genes and expressed sequence tags (ESTs). Sample preparation and microarray processing were performed using protocols from Affymetrix (Santa Clara, Calif., USA). In brief, total RNA was extracted from frozen whole blood using TRI REAGENT™ BD (Sigma, St. Louis, Mo., USA) and then purified through RNeasy Mini Spin Columns (Qiagen, Valencia, Calif., USA). Starting with 5-8 μg of purified total RNA, double-stranded cDNA was synthesized from full-length mRNA using Superscript Choice System (Invitrogen Life Technologies, Carlsbad, Calif., USA). The cDNA was then transcribed in vitro using BIOARRAY® High Yield RNA Transcript Labeling Kit (ENZO Diagnostics, Farmingdale, N.Y., USA) to form biotin-labeled cRNA. The cRNA was fragmented and hybridized to the microarrays for 16 hours at 45° C.

Arrays were washed and stained using an Affymetrix fluidics station according to standard Affymetrix protocols. Arrays were scanned using an Affymetrix GENEARRAY® scanner and the data (.DAT file) captured by the Affymetrix GENECHIP® Laboratory Information Management System (LIMS). The LIMS database was connected to an internal UNIX Sun Solaris server through a network filing system that allows for the average intensities for all probes cells (.CEL file) to be downloaded into an internal Oracle database. The fluorescence intensity of each microarray was normalized by global scaling to a value of 150 to allow for direct comparison across multiple arrays.

Quality of each array was assessed by evaluating factors such as background, percentage of genes present, scaling factor and the 3′/5′ ratios of the “housekeeping” genes β-actin and GAPDH. There was a wide range in these quality control parameters for the samples analyzed in this study and many samples were considered to be of sub-optimal quality. For example, the mean percent genes present for the 105 samples ranged from 5-38%, with a mean value of just 14%. This is approximately half of what has been observed from whole blood obtained from non-CML patients enrolled in other clinical trials. This discrepancy is likely due to problems with sample collection and handling, particularly the fact that the blood samples for this study were collected in EDTA tubes which contain no RNA stabilization factors, although it may also reflect a fundamental difference in overall gene expression in blood from leukemia patients. For the purposes of this Example, it is important to note that there were no statistically significant differences in sample quality between the Edema and No Edema groups, or between the “predictor” and “test” sets (data not shown).

Data Analysis

Starting with the 88-sample “predictor” set, the microarray data was imported into the GENESPRING® version 4.1.5 software (Silicon Genetics, San Carlos, Calif., USA). Raw expression values were filtered such that at least 10% of the samples (9 of 88) had an average intensity value of 100 or greater above background. Additional filtering steps were performed using GENESPRING®, Excel and SAS version 8.2 (The SAS Institute, Cary, N.C., USA) to identify a list of genes that most distinguished between the Edema and No Edema groups. A total of 88 genes fit the criteria of at least a 1.7-fold difference between the 2 groups with p<0.05 by non-parametric, one-way ANOVA. Lastly, 4 of these genes were eliminated after finding an association between expression levels and gender (using ANOVA of males vs. females for the No Edema group only). This was done in response to our finding that there was a significant association between the development of edema and gender for the patients in this study, with females being approximately 3 times more likely to develop edema following Imatinib treatment compared to males (p=0.022, Fisher's exact test).

From this list of 84 potential predictor genes, a “leave-one-out” procedure was employed to determine the optimum number of genes to use as the final prognostic set. See van't Veer, et al., Nature, Vol. 415, pp. 530-536 (2002). Genes were ordered by correlation (absolute value of PCC) between expression values and the prognostic category (0=No Edema; 1=Edema). Starting with the 5 most highly-correlated genes, one sample was taken out of the analysis and the mean gene expression profile for each group (Edema and No Edema) was calculated from the remaining 87 samples. The predicted outcome for the left-out sample was determined by comparing a PCC of the expression profile of the left-out sample with the mean Edema and No Edema profiles calculated using the 87 samples. This analysis was repeated using the remaining samples until all 88 samples had been left out once. The number of cases of correct and incorrect predictions was determined by calculating the number of false negatives (Edema misclassified as No Edema) and false positives (No Edema misclassified as Edema). The entire “leave-one-out” process was repeated after adding additional predictor genes, from the top of the list until all 84 genes were used. The gene number that resulted in the fewest false negatives and false positives was chosen as the optimal set of predictor genes (n=13).

The next step was to use this optimized set of genes to calculate an appropriate threshold value to use for an accurate prediction of Edema or No Edema. It was empirically decided to compare individual samples to the No Edema profile as opposed to the Edema profile after comparing results from both. A PCC was used to correlate the expression pattern of the predictor genes for each of the 88 samples to the mean No Edema profile (calculated using all 51 of the 88 patients with No Edema). Patient samples were ranked by correlation from highest to lowest and error rates were determined as a function of where the threshold correlation was drawn. The threshold at “optimal accuracy” was determined at the point where there was the minimum of both false positives and false negatives. However, to minimize the number of false negatives (Edema patients misclassified as having No Edema), a second threshold at “optimal sensitivity” that allowed for no more than 15% of Edema cases to be misclassified (5 of 37) was determined. Utilizing these threshold values, odds ratios (ORs) were calculated using SAS, with statistical significance determined by Fisher's exact test with a p-value cutoff of 0.05.

The final step was to validate the effectiveness of the selected predictor genes to predict edema status using the “test” set of 17 patient samples. The PCC for the predictor genes was calculated for each of these 17 samples against the mean No Edema expression profile from the 88-sample “predictor” set, and the threshold at “optimum sensitivity” was chosen as the cut-off for edema prediction. Thus, if the calculated for one of the 17 test samples was ≧threshold, that patient was categorized as having No Edema; if correlation was <threshold, the patient was predicted to have Edema. The OR and Fisher's exact test was performed based on the number of patients correctly and incorrectly predicted to have Edema or No Edema using SAS.

Results

Selection of the 13 genes used to predict Edema status was performed using a “predictor” set of 88 samples (37 Edema, 51 No Edema) as described in the Methods section. Table 2 presents a list of these genes along with their Affymetrix probe set name, GenBank Accession number, chromosomal locus, a brief description of function, as well as the fold difference between Edema and No Edema samples. Of the 13 genes, three are involved in signal transduction (PTPN12, P2Y10 and ARHGDIB), two are cell cycle regulators (CDKN1B and CUL1), two are involved immune response (FCER1G and MCP), two are involved in RNA processing (SFRS2IP and STAU), one is a transcription factor (HIVEP2), one is involved in metabolism (PGC), and two are of currently unknown function (CL24711 and FLJ00036). Expression of most of these genes is significantly higher in the Edema group as compared to No Edema, while two genes (PGC, P2Y10) are under-expressed in the Edema population.

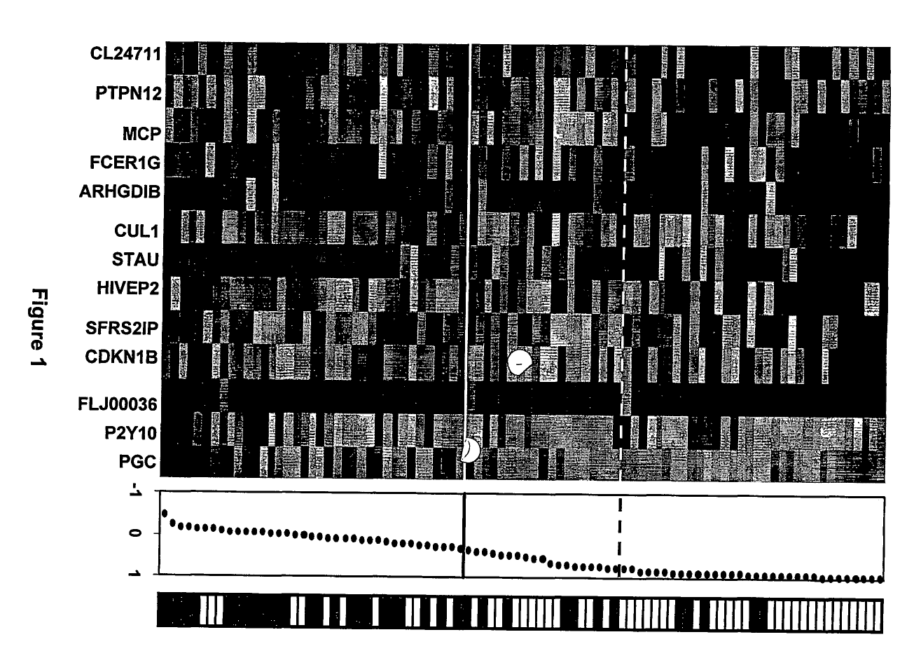

As discussed in the Methods section, a threshold value was determined by first ordering the samples in the predictor set according to their correlation with the mean No Edema expression profile. FIG. 1 displays the results of cluster analysis of these 13 genes, with the samples ordered by PCC, such that those samples with the highest correlation with the No Edema profile are at the top, and those with least correlation with No Edema status are at the bottom. As an initial starting point, threshold was determined at the point of optimal accuracy, which minimizes both the number of false positives (No Edema misclassified as Edema) and false negatives (Edema misclassified as No Edema). This occurred at a PCC value of 0.37 (FIG. 1, solid line). Using this threshold, an individual with a PCC ≧0.37 (based on PCC with mean No Edema expression profile) would be classified in the No Edema group, while an individual with a PCC <0.37 would be classified in the Edema group. The frequency of observations was determined and an OR calculated as shown in Table 4. The OR in this case indicates that a patient was 6.8 (95% CI: 2.6-17.4) times more likely to develop edema if their expression profile was negatively correlated with the mean No Edema profile (PCC <0.37). The difference between the observed and expected values was highly significant according to a Fisher's exact test, with a p-value of 6.25×10⁻⁵ (Table 4).

While these findings are statistically significant, it is important to note that 32% (12 of 37) of the Edema patients were actually misclassified as No Edema (false negatives). Since in rare instances edema can be a potentially life-threatening adverse event, it would be most clinically relevant in this case to minimize the number of false negatives so that patients could receive appropriate monitoring and treatment to help prevent the development of edema. This was the rationale for selecting the second threshold value at a PCC of 0.78. This value was optimized for sensitivity such that no more than 15% (5 of 37) of the Edema cases would be misclassified. Results of the frequency analysis using this criteria are presented in Table 4. Again, the difference between the observed and expected values was highly statistically significant, with a p-value of 1.37×10⁻⁴. The OR in this case indicates that a patient was 7.2 (95% CI: 2.4-21.4) times more likely to develop edema if their expression profile was negatively correlated with the mean No Edema profile (PCC <0.78).

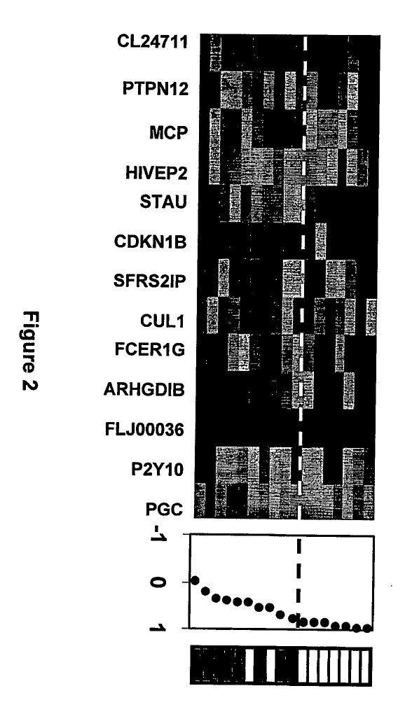

Validation of the effectiveness of the 13 predictor genes to predict Edema status was performed using the 17-sample “test” set of patients that were not included in the analysis to determine the predictor genes. These patients (8 Edema and 9 No Edema) were from the same clinical trial as the “predictor” set of patients. There were no significant differences in experimental parameters between the predictor and test sets (data not shown). Results of cluster analysis for this test set using the 13 predictor genes are presented in FIG. 2. Using the optimal sensitivity threshold of 0.78, enabled to correctly classify all of the 8 Edema patients, as well as 7 of the 9 No Edema patients, resulting in an overall accuracy of 88%. As demonstrated in the frequency analysis in Table 4, these results are statistically significant with a p-value of 0.0023 (Fisher's exact test). However, the OR of 51.0 is probably inflated due to small sample size.

The goal of this pharmacogenomic analysis was to identify genomic markers that could be used to predict susceptibility to Imatinib-induced edema and perhaps shed some light on the pathophysiology of edema. A total of 105 baseline blood samples from patients randomized to the Imatinib treatment arm were utilized for these analyses. Of these samples, a subset of 88 patients (37 Edema and 51 No Edema) were used as the “predictor” set to determine the list of predictor genes. The remaining 17 patients (8 Edema and 9 No Edema) were used as the “test” set to validate these predictor genes.

Utilizing the analytical strategy described by van't Veer et al. (2002) supra, enabled to define an optimal set of 13 genes to predict edema, and a threshold PCC value of 0.78 was chosen so as to minimize the number of false negatives. For the predictor set of samples, this resulted in an 86% success rate of identifying Edema patients (32 of 37), with an OR of 7.2 and p=1.37×10⁻⁴ (Table 4). This result was validated in the test set of 17 patients, with all of the 8 Edema patients correctly identified and overall prediction accuracy of 88%. These results were also statistically significant with a p-value of 0.0023, however the OR of 51.0, though significant, is likely inflated though due to the small sample size. Application of these results in at least one independent clinical trial, with enough patient samples to provide sufficient statistical power, should be performed to more substantially validate these preliminary findings.

As shown in Table 2, there is a diverse range of function for the 13 predictor genes. While two of the genes are of currently unknown function (CL24711 and FLJ0036), the remaining 11 genes have functions that include cell cycle regulation (CDKN1B and CULL), signal transduction (PTPN12, P2Y10 and ARHGDIB), RNA processing (SFRS21P and STAU), immune response (MCP and FCER1G), transcription factor (HIVEP2) and metabolism (PGC). Differential expression of these genes is predictive for Imatinib-induced edema.

TABLE 1
Occurrence of Edema in 105 Patients Treated with Imatinib

| Edema Classification | No. (%) |
|---|---|
| HLT: Angioedema | 35 (33.3) |
| PT: Periorbital oedema | 33 (31.4) |
| PT: Face oedema | 3 (2.9) |
| HLT: Edema NEC | 24 (22.9) |
| PT: Edema peripheral | 20 (19.0) |
| PT: Edema NOS | 3 (2.9) |
| PT: Pitting edema | 1 (1.0) |
| HLT: Pulmonary edemas | 1 (1.0) |
| PT: Pulmonary congestion | 1 (1.0) |
| ALL EDEMA CLASSES | 45 (42.9) |

HLT = MedDRA high level term.
PT = MedDRA preferred term.
NEC = not elsewhere classified.
NOS = not otherwise specified.

TABLE 2
Genes Used to Predict Development of Edema Following
Treatment with Imatinib

| Affymetrix | GenBank | Gene | | | | |
|---|---|---|---|---|---|---|
| Probe Set | Accession | Name | Locus | Description | Function | Fold |
| 34866_at | AF055029 | CL24711 | 2q21.2 | Homosapiens clone 24711 mRNA sequence | unknown | ↑2.4 |
| 36175_s_at | AL023584 | HIVEP2 | 6q23-q24 | Human immunodeficiency virus type I | Transcription factor | ↑2.9 |
| | | | | enhancer-binding protein 2 | | |
| 35258_f_at | AF030234 | SFRS2IP | 12q13.11 | Splicing factor, arginine/serine-rich 2, | RNA processing | ↑2.3 |
| | | | | interacting protein | | |
| 33848_r_at | AI304854 | CDKN1B | 12p13.1-p12 | Cyclin-dependent kinase inhibitor 1B | Cell cycle regulator | ↑2.0 |
| | | | | (p27, Klp1) | | |
| 1463_at | M93425 | PTPN12 | 7q11.23 | Protein tyrosine phosphatase, non-receptor | Signal transduction | ↑2.0 |
| | | | | type 12 | | |
| 39724_s_at | U58087 | CUL1 | 7q36.1 | Cullin 1 | Cell cycle regulator | ↑2.0 |
| 41823_at | AJ132258 | STAU | 20q13.1 | Staufen (Drosophila, RNA-binding protein) | RNA processing | ↑2.3 |
| 38732_at | AI004207 | SIMRP7 | 6p21.31 | multidrug resistance-associated protein 7 | Unknown | ↑3.0 |
| 358_at | AF000545 | P2Y10 | Xq21.1 | Putative purinergic receptor | Signal transduction | ↓1.7 |
| 38441_s_at | X59408 | MCP | 1q32 | Membrane cofactor protein (CD46, | Immune response | ↑1.9 |
| | | | | trophoblast-lymphocyte cross-reactive | | |
| | | | | antigen) | | |
| 36889_at | M33195 | FCER1G | 1q23 | Fc fragment of IgE, high affinity I, receptor | Immune response | ↑2.4 |
| | | | | for; gamma polypeptide | | |
| 33699_at | M18667 | PGC | 6p21.3-p21.1 | Progastricsin (pepsinogen C) | Metabolism | ↓2.5 |
| 1984_s_at | X69549 | ARHGDIB | 12p12.3 | Rho GDP dissociation inhibitor (GDI) beta | Signal transduction | ↑2.4 |

Note:
Genes determined from “leave-one-out” analysis of 84 potential candidate genes using “predictor” set of 88 patient samples (37 Edema, 51 No Edema). Genes ordered by absolute correlation with Edema status, from highest to lowest.
Fold = Fold difference (Edema vs. No Edema group).

TABLE 3
Mean No Edema and Edema Expression Profiles For the 13 Predictor Genes

| Affymetrix | GenBank | | | No | |
|---|---|---|---|---|---|
| Probe Set | Accession | Gene Name | Description | Edema | Edema |
| 34866_at | AF055029 | CL24711 | Homo sapiens clone 24711 mRNA sequence | 56.1 | 134.4 |
| 36175_s_at | AL023584 | HIVEP2 | Human immunodeficiency virus type I enhancer-binding | 48.5 | 139.9 |
| | | | protein 2 | | |
| 35258_f_at | AF030234 | SFRS2IP | Splicing factor, arginine/serine-rich 2, interacting protein | 53.4 | 124.6 |
| 33848_r_at | AI304854 | CDKN1B | Cyclin-dependent kinase inhibitor 1B (p27, Klp1) | 48.4 | 98.4 |
| 1463_at | M93425 | PTPN12 | Protein tyrosine phosphatase, non-receptor type 12 | 156.0 | 306.2 |
| 39724_s_at | U58087 | CUL1 | Cullin 1 | 67.2 | 133.4 |
| 41823_at | AJ132258 | STAU | Staufen (Drosophila, RNA-binding protein) | 40.4 | 91.1 |
| 36732_at | AI004207 | SIMRP7 | Multidrug resistance-associated protein 7 | 80.5 | 239.4 |
| 358_at | AF000545 | P2Y10 | Putative purinergic receptor | 342.8 | 198.6 |
| 38441_s_at | X59408 | MCP | Membrane cofactor protein (CD46, trophoblast- | 108.6 | 206.7 |
| | | | lymphocyte cross-reactive antigen) | | |
| 36889_at | M33195 | FCER1G | Fc fragment of IgE, high affinity I, receptor for; gamma | 91.1 | 219.2 |
| | | | polypeptide | | |
| 33699_at | M18667 | PGC | Progastricsin (pepsinogen C) | 606.9 | 331.4 |
| 1984_s_at | X69549 | ARHGDIB | Rho GDP dissociation inhibitor (GDI) beta | 118.6 | 290.3 |

TABLE 4
Frequency Analysis and Calculation of ORs

| Observed (Expected) | | | |
|---|---|---|---|
| PCC* ≧ Threshold | PCC* < Threshold | | |
| (No Edema) | (Edema) | OR (95% CI) | p-value |

Predictor Set (Thr = 0.37)

| | | | | |
|---|---|---|---|---|
| Edema | 12 (21.4) | 25 (15.6) | 6.8 (2.6-17.4) | 6.25 × 10⁻⁵ |
| No Edema | 39 (29.6) | 12 (21.4) | | |
| Predictor Set (Thr = 0.78) | | | | |
| Edema | 5 (13.5) | 32 (23.6) | 7.2 (2.4-21.4) | 1.37 × 10⁻⁴ |
| No Edema | 27 (18.6) | 24 (32.5) | | |
| Test Set (Thr = 0.37) | | | | |
| Edema | 6 (7.1) | 2 (0.9) | 7.3 (0.3-178) | 0.206 |
| No Edema | 9 (7.9) | 0 (1.1) | | |
| Test Set (Thr = 0.78) | | | | |
| Edema | 0 (3.3) | 8 (4.7) | 51.0 (2.1-1240) | 0.0023 |
| No Edema | 7 (3.7) | 2 (5.3) | | |

*Compared to mean No Edema expression profile for the 13 predictor genes.
p-value calculated using Fisher's exact test.
Predictor Set = 88 patient samples used to determine list of predictor genes.
Test Set = 17 patient samples used to validate predictor genes.

### EXAMPLE 2

Polymorphisms in the IL-1β Gene

Pharmacogenetic analysis was conducted to identify genetic factors that associate with the adverse event of edema in a Phase III Clinical Trial. Seventy SNPs from 26 genes were examined in a 6-month interim analysis and a significant association between periorbital and face edema and the −511 T→C polymorphism in the IL-1β gene in Imatinib treated individuals was observed (p=0.016, OR: 3.06, 95% CI: 1.29-7.27). The same analysis was done stratifying by gender. A significant association was found between periorbital and face edema and the IL-1β polymorphism in women (p=0.0005574). Women with a CC genotype for the −511 polymorphism are 13.0 times more likely to experience edema then Imatinib-treated females with a non-CC genotype (95% CI: 2.07-81.48) (from 12-month locked data). No association was observed in men. Therefore the association of the −511 IL-1β polymorphism with edema appears to be specific to females and may explain why women are three times as likely as men to experience edema when treated with Imatinib. The results of this study suggest the −511 polymorphism in the IL-1β promoter can be used as a predictive marker of periorbital and face edema in Imatinib-treated females.

Pharmacogenetic analysis to identify predictive markers of the adverse event edema was conducted in a clinical trial. This was a Phase III study of Imatinib vs. IFN-α combined with Ara-C in patients with newly diagnosed, previously untreated Ph⁺ CML-CP. Genotypes for 151 patients treated with Imatinib or IFN-α were analyzed. A total of 57.72% of U.S. patients consented to participate in this pharmacogenetic analysis.

The “Briefing Book on the etiology and proposed investigation strategy for edema and fluid retention in patients treated with Gleevec” summarized a trend of higher frequency of edema in certain groups of patients within the GLEEVEC® Imatinib clinical trials. These groups included elderly patients (65 years and above), patients with higher area under the curve (AUC) values, and patients with advanced stages of CML. In this same report five covariates; age (above 65 years), history of cardiovascular disease, females, advanced phase of CML and patients with double the values for the average concentration of Imatinib at steady-state, were reported to significantly associate with Grade 34 edema.

Thus was identified a significant association between the −511 polymorphism in the promoter of the IL-1β gene and periorbital and face edema. Due to the associations with edema and demographic factors outlined in the Briefing Book on edema, the demographic factors within the Imatinib study population with respect to this association were examined. The analyses was stratified by gender and discovered a significant association in Imatinib-treated females between the −511 IL-1β genotype and periorbital and face edema (p=0.0005574). There was no association in males; therefore the association appears to be gender specific. The identification of IL-1β as a predictive marker could aid physicians in the treatment of female patients with CML and prevention of severe edema.

A candidate gene approach was used to identify genetic polymorphisms that could be used as predictive markers of edema and might suggest a mechanism of action for edema formation. SNPs were developed by two distinct methods. Third Wave Technologies, Inc. developed one collection of SNPs while the other set was developed in-house using a database mining approach. Public databases, such as OMIM, the SNP Consortium, Locus Link and dbSNP were utilized. Candidate genes were chosen based on rationale that included their involvement in edema, DNA repair, etiology of the disease or drug mechanism of action. Third Wave Technologies, Inc. developed the SNP assays for genotyping.

Genotyping

On the first day of study before treatment administration, 20 mL of blood was obtained from patients enrolled in the U.S. only. The blood samples were collected after informed consent had been obtained according to protocols approved by local ethics committees. The DNA was extracted using the PUREGENE™ DNA Isolation Kit (D-50K) (Gentra, Minneapolis, Minn.) according to manufacturer's recommendations. Genotypic and phenotypic data was evaluated for a total of 151 patients. Genotyping was performed on 60 ng of genomic DNA using the Invader® assay (Third Wave Technologies, Inc.) according to the manufacturers recommendations. See Lyamichev et al., Nat Biotechnol., Vol. 17, pp. 292-296 (1999).

Those SNPs that were significantly associated with edema were genotyped a second time in the laboratory to confirm the genotypes. An additional quality control check was performed; genotypes were tested for Hardy Weinberg Equilibrium (HWE). The HWE law states that allele frequencies do not change from generation to generation in a large population with random mating. Deviation from HWE would suggest one of two possibilities:
1) a genotyping error; or
2) an association between the polymorphism and the population being studied.
   In the second case you might see a particular polymorphism more predominantly than would be expected if it is somehow involved in the disease etiology. For example, in a study of Alzheimer's Disease (AD) patients apolipoprotein E (APOE) may not be in HWE because APOE ε4 pre-disposes patients to develop AD. All statistics were carried out in the statistical program SAS version 8.2.
   Gene Expression Profiling

Blood samples were processed for Gene Expression Profiling of Whole Blood Using TRI REAGENT™ BD. RNA was extracted from 470 blood samples, preserved at −80° C. In this study 96 expression profiles from patients for whom there was also genotype information for were examined.

Statistical Methods

Representative Nature of the Genotyped Population

To determine how representative the genotyped population was of the entire clinical trial population, demographics and occurrence of edema in the two populations was compared. Furthermore, because the genotyped population consisted solely of U.S. patients, all U.S. patients in the trial as an additional population were examined. Age was compared using a non-parametric ANOVA and all others were analyzed using Fisher's exact tests in the statistical program SAS version 8.2.

Correlation of Genotype with Edema Status

A Fisher's exact test was used to compare the genotype of each patient to the clinical phenotype of Edema status. Edema status was determined from the clinical database, which compiled data following a minimum of 12 months of treatment. The mechanism of edema in Imatinib-treated patients is unknown. Furthermore, it is unknown whether the more severe fluid retention events have the same pathophysiology as the more common periorbital and peripheral edema events. In the patients studied, one patient experienced a Grade 3 severe periorbital edema event. The vast majority experienced only one of the milder events or no fluid retention at all. Consequently, as few assumptions regarding the pathophysiology of edema as was feasible in our statistical association studies were made. Three analyses of association between genotype and edema were performed. The first consisted of patients with any form of edema (55% of cases analyzed) compared to all other subjects without edema. The second and third analyses were performed using the sub-groups with the highest incidence of edema. For example, Group 1 (periorbital edema and face edema) was classified as having Edema and all other patients as having No Edema. Likewise, the third analysis coded Group 2 individuals (edema peripheral, edema NOS and pitting edema) as having Edema and all others as having No Edema.

Each genotype/phenotype correlation was stratified by treatment because it was not expect to see similar results with Imatinib and IFN-α and were primarily interested in the Imatinib results. The number of patients used in the final analysis was 91 Imatinib-treated patients. All statistics were carried out in the statistical program SAS Version 8.2.

Logistic Regression

Logistic regression was employed to determine which variables are predictive of periorbital and face edema. Periorbital/face edema was the dependent variable utilized in the various models. The models were used to allow any confounding effects of genotype and demographic factors to be taken into consideration. The logistic regression was constructed to model the original association observed between −511 IL-1β genotype and edema. All models consisted of both males and females treated with Imatinib (n=91). In the first logistic regression analysis age, sex, race and −511 IL-1β genotype were added as classes to the full model. In order to investigate the possibility of an interaction between sex and genotype, as previously observed, an additional variable to allow a 2-way covariate interaction was created. Additional analyses consisted of the classes utilized in the first model, along with two additional variables that allowed 3-way interactions between genotype, sex and race, and between genotype, sex and age. Due to the low prevalence of Black, Oriental and other individuals, the racial groups were transformed into two categories, Caucasian and other, and completed the logistic regression a third time as described above. The logistic regression was completed as an exploratory analysis to further characterize the significant genotype/phenotype correlations.

The polymorphism was analyzed in the 91 Imatinib patients reported here plus an additional 18 IFN-α-treated patients and was found to be in HWE. The −511 IL-1β polymorphism lies in the promoter region of the gene and represents a C/T base transition at the −511 base pairs upstream from the transcriptional start site. See El-Omar et al., Nature, Vol. 404, pp. 389-402 (2000). Due to the near-complete linkage disequilibrium (LD) of the −511 IL-1β polymorphism with the −31 variant in the same gene, the −31 IL-1β polymorphism was genotyped and an association between it and Edema in Imatinib-treated females was also observed (p=0.0054).

Table 5, below, shows the Edema vs. No Edema in Imatinib-treated females according to IL-1β genotype. This table displays the distribution of genotypes for the two IL-1β polymorphisms associated with Edema in Imatinib-treated females. Females are characterized according to whether or not they experienced edema as an adverse event. There are significantly more females with a CC genotype at the −511 locus and a TT at the −31 locus who experienced edema then with the alternative genotypes at this loci (p=0.0041 and p=0.0054, respectively).

TABLE 5

| | −511 | −511 | −511 | −31 | −31 | −31 |
|---|---|---|---|---|---|---|
| | CC | CT | TT | TT | CT | CC |
| No edema | 2 | 12 | 1 | 1 | 12 | 2 |
| Edema | 10 | 4 | 1 | 7 | 3 | 2 |

LD of the −511 and −31 IL-1β SNPs

D′, a statistic used to calculate the degree of LD, was computed to confirm the report of near-complete LD between the −511 and −31 IL-1β promoter polymorphisms. D′ has the same range of values regardless of the frequencies of the two polymorphisms that are being compared. The EH linkage utility program was used to test and estimate LD between the two markers. On the basis of the sample data taken to consist of a number of individuals in a population collected at random, the EH program estimates allele frequencies for each marker. See Xie et al., Am. J. Hum. Genet., Vol. 53, p. 1107 (abstract) (1993); and Terwilliger et al., John's Hopkins University Press, Baltimore (1994).

The −511 IL-1β polymorphism and the −31 IL-1β polymorphism are in near-complete LD in this population of CML patients with a |D′| of 0.978, computed by the EH program. See Xie et al., supra. A |D′| value of 1 indicates complete LD, whereas a |D′| value of zero suggests no LD. See Reich et al., Nature, Vol. 411, pp. 199-204 (2001). Therefore, all statistically significant associations that are observed with one IL-1β polymorphism would also be statistically significant with the alternative IL-1β polymorphism. Since the IL-1β (−511) C→T polymorphism is in strong LD (99.5%) with another polymorphism within the IL-1β promoter located at position (−31) that results in a T→C base transition. See El-Omar et al. (2000), supra, therefore, it is predicted that patients with a T at position (−511) of the IL-1β promoter would have a C at position (−31). This finding was confirmed in the patients tested in these two trials. In the wild-type IL-1β gene, T is found at position at −31. This T is very important for the expression of IL-1β because it is part of the TATA box sequence (TATAAAA) which plays a critical role in the transcriptional initiation of IL-1β. In general, TATA box sequences are involved in recruiting and positioning the transcriptional machinery at the correct position within genes to ensure that transcription begins at the correct place. The T→C polymorphism at position (−31) would disrupt this important TATA box sequence (TATAAAA to CATAAAA), thus making it inactive and prohibiting the efficient initiation of transcription of the IL-1β gene. The lack of binding of the transcriptional machinery to this altered IL-1β TATA box sequence has been shown. See El-Omar (2002), supra. Conversely, the C polymorphism at −511 is correlated with the T at −31. Thus women with an intact TATA box in the promoter may be at greater risk of drug induces edema. Therefore, the existence of any other polymorphism which is in LD with either the polymorphism within the IL-1β promoter (located at position (−31) that results in a T→C base transition) or the polymorphism located at −511 (C→T) of the IL-1β promoter, would also have a predictive effect on the likelihood of the development of edema with drug treatment. The means for the determination of other polymorphisms which are in LD with the (−31) polymorphism is well-known to one of skill in the art. Any such polymorphism, now known or discovered in the future, could be used in the methods of this invention to predict the likelihood edema formation in a patient when treated with a drug or to help determine treatment choices for such.

Correlation Analysis Between Demographic, Genotypic and Phenotypic Variables

The genetic makeup of individuals from diverse ethnic groups vary greatly. In order to assess this variance each polymorphism was analyzed by race. Also investigated was whether there was any difference in the occurrence of edema between races. In study data panel, race was classified as Caucasians, Blacks, Orientals and Others. The number of non-Caucasians was small. To increase the power of the analysis the analyses with race re-coded as Caucasians and non-Caucasians was also performed. P-values in this portion of the analysis were calculated using Fisher's exact tests. All statistics were carried out in the statistical program SAS version 8.2.

In addition to race it was also investigated whether sex and/or age were associated with edema and whether these variables were independent of the associations with the −511 IL-1β SNP. Sex and age were examined because our previous experience suggested that they were associated with angioedema. Sex and age were determined from the study data set. All of the associations studies between edema phenotype and the associated SNPs were stratified by sex. A one-way ANOVA between age and all classes of edema was performed. Logistic regression was employed to determine which variables are predictive of periorbital and face edema. Periorbital/face edema was the dependent variable utilized in the various models. The models were used to allow any confounding effects of genotype and demographic factors to be taken into consideration. The logistic regression was constructed to model the original association observed between −511 IL-1β genotype and edema.

All models consisted of both males and females treated with Imatinib (n=91). In the first logistic regression analysis age, sex, race and −511 IL-1β genotype were added as classes to the full model. In order to investigate the possibility of an interaction between sex and genotype, as previously observed, an additional variable to allow a 2-way covariate interaction was created. Additional analyses consisted of the classes utilized in the first model, along with two additional variables that allowed 3-way interactions between genotype, sex and race, and between genotype, sex and age. Due to the low prevalence of Black, Oriental and Other individuals, the racial groups were transformed into two categories, Caucasian and Other, and completed the logistic regression a third time as described above. The logistic regression was completed as an exploratory analysis to further characterize the significant genotype/phenotype correlations.

The OR and 95% CIs were calculated by dividing the odds of having a particular genotype (for example, CC vs. non-CC) in the Edema group by the odds of having that same genotype in the No Edema group.

Correction for Multiple Testing

Because of the nature of the approach used to identify predictive markers of edema, it must be corrected for multiple testing. The more tests performed, the greater the chance of finding an association with p<0.05 by chance. To correct for multiple testing by using the Bonferroni correction factor the desired p-value is divided by the number of tests performed. The resulting value is the value that would be considered “significant”. So, for the 70 polymorphisms tested in this analysis a p-value of 0.0007 would be required to be considered significant. This is an extremely small number and it is likely that with this conservative cut off potentially useful predictive markers would be missed.

A second method of correcting for multiple testing is bootstrapping. This method is a computer-intensive statistical analysis that applies simulation to calculate significance tests. A random number generator is utilized to resample the dataset. Bootstrapping was performed to test the stability of our significant results. The bootstrap consisted of the edema phenotype and 68 polymorphisms and was run with 10,000 iterations using females only. All statistics were carried out in the statistical program SAS version 8.2.

Representative Nature of the Genotyped Population

To determine whether the subset of patients that were used for pharmacogenetic studies in the study were representative of the trial population several demographics relevant to edema were examined. The genotyped population consisted of patients from the U.S. only. Therefore, the genotyped population to the entire U.S. patient population of the clinical trial was also compared. The genotyped population of the study is similar to the trial population from the U.S. with regards to sex, race, age and development of edema. They differ only with regards to race. The U.S. population has more Blacks, 11.92%, compared to 4.78%, fewer Caucasians, 80.13% compared to 90.06% and slightly more individuals in the other category, 6.62% vs. 3.50%.

Results of Correlation with Genotype and Edema Status

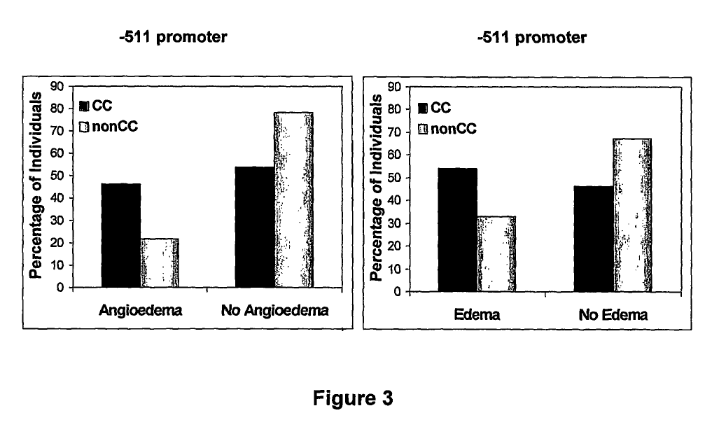

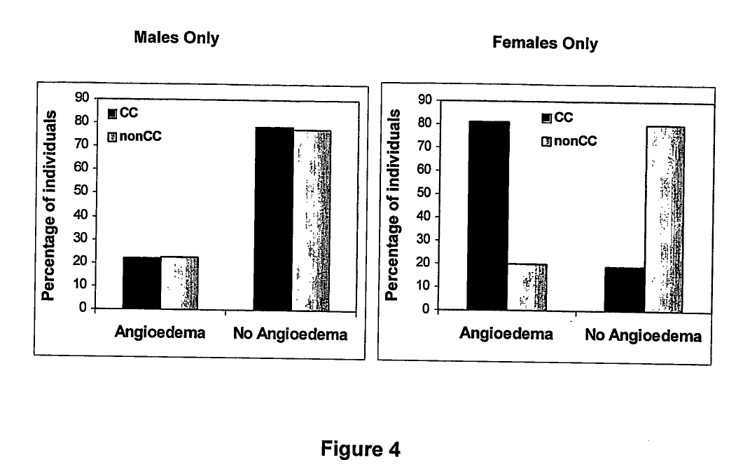

Analysis of 70 genetic polymorphisms in 26 genes identified the −511 polymorphism of the IL-1β gene to be significantly associated with periorbital and face edema in Imatinib-treated females, p=0.00056. Females who are of the CC genotype are 13.0 times more likely to develop periorbital and face edema than individuals of the non-CC genotype (95% CI: 2.07-81.48); see also FIG. 3 and FIG. 4 The IL-1β polymorphism lies in the promoter region and represents a C→T base transition at position −511 base pairs upstream from the transcriptional start site.

Results of Correlations Between Demographic, Genotypic and Phenotypic Variables

Sex and age were found to be significantly associated with angloedema in Imatinib-treated individuals (p<0.05). Surprisingly, sex is associated with the IL-1β polymorphism in all trial patients (p=0.0106), FIG. 4. Females who are of the CC genotype are more likely to develop angioedema than females of the non-CC genotype. An association with sex and a genetic polymorphism is unexpected because the IL-1β gene lies on an autosome. In an attempt to understand whether the sex association with the −511 polymorphism was specific to the study trial population or observed in other control populations, three additional non-related clinical trials were examined. No significant association between sex and the −511 IL-1β SNP was observed for all trials combined, nor for any of the three control trials. The genotype distributions for the −511 IL-1β polymorphism were not significantly different between Imatinib and all others. However, when the analysis was stratified by sex there was a significant difference in the genotype distribution in females from the study trial compared to all other females. It appears that there is an absence of female leukemia patients with TT genotype for the −511 IL-1β polymorphism. The distribution of CC:CT:TT genotypes in Imatinib-treated females is 16:19:1 compared to males who were 23:25:19, respectively. There was not a significant difference in distribution of males from the study compared to all other trials.

The genotype distribution is significantly different among the four races for this polymorphism. To investigate whether the observed association between IL-1β and angloedema was race specific the analysis by race was stratified. In Imatinib-treated males and females the −511 IL-1β CC:CT:TT distribution in Blacks is 1:6:4, Caucasians 29:31:13, Orientals 0:2:0, Others 3:0:2, and in all groups combined 33:39:19. When the data is stratified by sex and race Caucasians make up 77% of the women studied. There is a clear trend in Caucasians that the CC genotype for the −511 IL-1β polymorphism predispose women to edema when treated with Imatinib. Future studies should include the appropriate number of individuals from different racial backgrounds to determine whether the −511 IL-1β polymorphism association with edema is race specific. The difference in allele distribution for the IL-1β polymorphism observed between the four different races characterized could result in differences in the incidence of edema between races.

Age was associated with angioedema, non-parametric ANOVA p=0.0393. However, it was not associated with the IL-1β polymorphism suggesting that it is an independent variables in the development of angioedema.

Correction for Multiple Testing

Bonferroni Correction

A correction for multiple testing due to the number of SNPs analyzed and the fact that numerous tests may introduce false positive error rates was performed. The finding of the associations with edema and the IL-1β variants in Imatinib-treated females would not be considered significant using the Bonferroni correction method which dictates a p-value of 0.0007 as calculated below.

$$Bonferroni = \frac{0.05}{η} = \frac{0.05}{68} = 0.0007$$

η=number of tests

The p-values observed with the −511 and −31 polymorphisms were greater than the 0.0007 cut-off.

Bootstrapping

The bootstrap analysis resulted in a corrected p-value of 0.058 for the association between edema and the −511 IL-1β polymorphism in females treated with Imatinib.

A pharmacogenetic analysis was performed to identify genetic markers that could be used to predict susceptibility to Imatinib-induced edema and ideally assist in understanding the pathophysiology of edema. Statistical tests to look for associations between genotypes in candidate genes and the presence of edema in patients from the study trial was performed. An association was discovered between the −511 IL-1β polymorphism and periorbital and face edema in Imatinib-treated females only. A female patient treated with Imatinib with a CC genotype at the IL-1β −511 locus is 13.0 times more likely to experience angioedema than Imatinib-treated females with a CT or TT genotype (95% CI: 2.07-81.48). Therefore, a surrogate marker for periorbital and face edema in the IL-1β gene that accounts for 67% (10 out of 15) of the observed cases in Imatinib-treated females has been identified. It is likely that the genotype associated with angioedema functionally relates to a increased level of expression of the IL-1β gene. Such a surrogate marker could easily be applied in the clinic to predict a patient's susceptibility to angioedema so that they might get closer monitoring or preventative therapies. The test could be genetic or potentially a measurement of IL-1β protein levels in serum.

As used herein, the term “Edema” shall refer to the occurrence of any type or kind of clinically significant edema including, but not limited to, angioedema, including periorbital edema and face edema, edema NEC including edema peripheral, edema NOS and pitting edema and pulmonary edemas including pulmonary congestion and cerebral edema.

As used herein, the term “No Edema” shall mean the absence of clinically significant edema of any type or kind.

Microarray Technology in General

Microarray technology that evaluates the signatures of thousands of individual genes at a time is growing rapid acceptance in the clinical oncology setting. This technology has been utilized to identify genetic factors that can differentiate between different classes of cancers, biomarkers of clinical response, as well as genes that can predict the development of resistance to Imatinib treatment in cases of acute lymphoblastic leukemia. The goal of this study was to utilize this gene expression profiling strategy to identify predictive gene expression profiles of edema in CML patients treated with Imatinib.

Measurement Methods

The experimental methods of this invention depend on measurements of cellular constituents. The cellular constituents measured can be from any aspect of the biological state of a cell. They can be from the transcriptional state, in which RNA abundances are measured, the translation state, in which protein abundances are measured, the activity state, in which protein activities are measured. The cellular characteristics can also be from mixed aspects, for example, in which the activities of one or more proteins are measured along with the RNA abundances (gene expressions) of other cellular constituents. This section describes exemplary methods for measuring the cellular constituents in drug or pathway responses. This invention is adaptable to other methods of such measurement.

Preferably, in this invention the transcriptional state of the other cellular constituents is measured. The transcriptional state can be measured by techniques of hybridization to arrays of nucleic acid or nucleic acid mimic probes, described in the next subsection, or by other gene expression technologies, described in the subsequent subsection. However measured, the result is data including values representing mRNA abundance and/or ratios, which usually reflect DNA expression ratios (in the absence of differences in RNA degradation rates).

In various alternative embodiments of the present invention, aspects of the biological state other than the transcriptional state, such as the translational state, the activity state or mixed aspects can be measured.

Cell-free assays can also be used to identify compounds which are capable of interacting with a protein encoded by one of the disclosed genes in Table 2 or protein binding partner, to alter the activity of the protein or its binding partner. Cell-free assays can also be used to identify compounds, which modulate the interaction between the encoded protein and its binding partner such as a target peptide.

Interaction between molecules can also be assessed by using real-time Biomolecular Interaction Analysis (BIA) Pharmacia Biosensor (AB) which detects surface plasmon resonance, an optical phenomenon. Detection depends on changes in the mass concentration of mass macromolecules at the biospecific interface and does not require labeling of the molecules. In one useful embodiment, a library of test compounds can be immobilized on a sensor surface, e.g., a wall of a micro-flow cell. A solution containing the protein, functional fragment thereof, or the protein binding partner is then continuously circulated over the sensor surface. An alteration in the resonance angle, as indicated on a signal recording, indicates the occurrence of an interaction. This technique is described in more detail in “BIAtechnology Handbook” by Pharmacia.

Another embodiment of a cell-free assay comprises:
a) combining a protein encoded by the at least one gene, the protein binding partner and a test compound to form a reaction mixture; and
b) detecting interaction of the protein and the protein binding partner in the presence and absence of the test compounds.

A considerable change (potentiation or inhibition) in the interaction of the protein and binding partner in the presence of the test compound compared to the interaction in the absence of the test compound indicates a potential agonist (mimetic or potentiator) or antagonist (inhibitor) of the proteins' activity for the test compound. The components of the assay can be combined simultaneously or the protein can be contacted with the test compound for a period of time, followed by the addition of the binding partner to the reaction mixture. The efficacy of the compound can be assessed by using various concentrations of the compound to generate dose response curves. A control assay can also be performed by quantitating the formation of the complex between the protein and its binding partner in the absence of the test compound.

Formation of a complex between the protein and its binding partner can be detected by using detectably-labeled proteins such as radiolabeled, fluorescently-labeled or enzymatically-labeled protein or its binding partner, by immunoassay or by chromatographic detection.

In preferred embodiments, the protein or its binding partner can be immobilized to facilitate separation of complexes from uncomplexed forms of the protein and its binding partner and automation of the assay. Complexation of the protein to its binding partner can be achieved in any type of vessel, e.g., microtitre plates, micro-centrifuge tubes and test tubes. In particularly preferred embodiment, the protein can be fused to another protein, e.g., glutathione-S-transferase to form a fusion protein which can be absorbed onto a matrix, e.g., glutathione sepharose beads (Sigma Chemical, St. Louis, Mo.) which are then combined with the labeled protein partner, e.g., labeled with ³⁵S, and test compound and incubated under conditions sufficient to formation of complexes. Subsequently, the beads are washed to remove unbound label and the matrix is immobilized and the radiolabel is determined.

Another method for immobilizing proteins on matrices involves utilizing biotin and streptavidin. For example, the protein can be biotinylated using biotin N-hydroxy-succinimide using well-known techniques and immobilized in the well of steptavidin-coated plates.

Cell-free assays can also be used to identify agents which are capable of interacting with a protein encoded by the at least one gene and modulate the activity of the protein encoded by the gene. In one embodiment, the protein is incubated with a test compound and the catalytic activity of the protein is determined. In another embodiment, the binding affinity of the protein to a target molecule can be determined by methods known in the art.

As used herein the term “antisense” refers to nucleotide sequences that are complementary to a portion of an RNA expression product of at least one of the disclosed genes. “Complementary” nucleotide sequences refer to nucleotide sequences that are capable of base-pairing according to the standard Watson-Crick complementary rules. That is, purines will base-pair with pyrimidine to form combinations of guanine:cytosine and adenine:thymine in the case of DNA, or adenine:uracil in the case of RNA. Other less common bases, e.g., inosine, 5-methylcytosine, 6-methyladenine, hypoxanthine and others may be included in the hybridizing sequences and will not interfere with pairing.

In all embodiments, measurements of the cellular constituents should be made in a manner that is relatively independent of when the measurements are made.

Transcriptional State Measurement

Preferably, measurement of the transcriptional state is made by hybridization of nucleic acids to oligonucleotide arrays, which are described in this subsection. Certain other methods of transcriptional state measurement are described later in this subsection.

Transcript Arrays Generally

In a preferred embodiment, the present invention makes use of “oligonucleotide arrays” (also called herein “microarrays”). Microarrays can be employed for analyzing the transcriptional state in a cell, and especially for measuring the transcriptional states of cancer cells.

In one embodiment, transcript arrays are produced by hybridizing detectably-labeled polynucleotides representing the mRNA transcripts present in a cell (e.g., fluorescently-labeled cDNA synthesized from total cell mRNA or labeled cRNA) to a microarray. A microarray is a surface with an ordered array of binding (e.g., hybridization) sites for products of many of the genes in the genome of a cell or organism, preferably most or almost all of the genes. Microarrays can be made in a number of ways, of which several are described below. However produced, microarrays share certain characteristics: The arrays are reproducible, allowing multiple copies of a given array to be produced and easily compared with each other. Preferably the microarrays are small, usually smaller than 5 cm², and they are made from materials that are stable under binding (e.g., nucleic acid hybridization) conditions. A given binding site or unique set of binding sites in the microarray will specifically bind the product of a single gene in the cell. Although there may be more than one physical “binding site” (hereinafter “site”) per specific mRNA, for the sake of clarity the discussion below will assume that there is a single site. In a specific embodiment, positionally addressable arrays containing affixed nucleic acids of known sequence at each location are used.

It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably-labeled (e.g., with a fluorophore) cDNA or cRNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

Preparation of Microarrays

Microarrays are known in the art and consist of a surface to which probes that correspond in sequence to gene products (e.g., cDNAs, mRNAs, cRNAs, polypeptides and fragments thereof), can be specifically hybridized or bound at a known position. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the site is a nucleic acid or nucleic acid analogue to which a particular cognate cDNA or cRNA can specifically hybridize. The nucleic acid or analogue of the binding site can be, e.g., a synthetic oligomer, a full-length cDNA, a less-than full-length cDNA, or a gene fragment.

Although in a preferred embodiment the microarray contains binding sites for products of all or almost all genes in the target organism's genome, such comprehensiveness is not necessarily required. The microarray may have binding sites for only a fraction of the genes in the target organism. However, in general, the microarray will have binding sites corresponding to at least about 50% of the genes in the genome, often at least about 75%, more often at least about 85%, even more often more than about 90% and most often at least about 99%. Preferably, the microarray has binding sites for genes relevant to testing and confirming a biological network model of interest. A “gene” is identified as an open reading frame (ORF) of preferably at least 50, 75 or 99 amino acids from which a mRNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence. For example, the Saccharomyces cerevisiae genome has been completely sequenced and is reported to have approximately 6,275 ORFs longer than 99 amino acids. Analysis of these ORFs indicates that there are 5,885 ORFs that are likely to specify protein products, see Goffeau et al., “Life with 6000 Genes”, Science, Vol. 274, pp. 546-567 (1996), which is incorporated by reference in its entirety for all purposes. In contrast, the human genome is estimated to contain approximately 25,000-35,000 genes.

Preparing Nucleic Acids for Microarrays

As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., PCR amplification of gene segments from genomic DNA, cDNA (e.g., by RT-PCR), or cloned sequences or the sequences may be synthesized de novo on the surface of the chip, for example by use of photolithography techniques, e.g., Affymetrix uses such a different technology to synthesize their oligos directly on the chip). PCR primers are chosen, based on the known sequence of the genes or cDNA, that result in amplification of unique fragments (i.e., fragments that do not share more than 10 bases of contiguous identical sequence with any other fragment on the microarray). Computer programs are useful in the design of primers with the required specificity and optimal amplification properties. See, e.g., Oligo, pI version 5.0, Nat. Biosci. In the case of binding sites corresponding to very long genes, it will sometimes be desirable to amplify segments near the 3′ end of the gene so that when oligo-dT primed cDNA probes are hybridized to the microarray; less than full-length probes will bind efficiently. Typically each gene fragment on the microarray will be between about 20 bp and about 2000 bp, more typically between about 100 bp and about 1000 bp, and usually between about 300 bp and about 800 bp in length. PCR methods are well-known and are described, for example, in Innis et al., Eds., PCR Protocols: A Guide to Methods and Applications, Academic Press Inc., San Diego, Calif. (1990), which is incorporated by reference in its entirety for all purposes. It will be apparent that computer controlled robotic systems are useful for isolating and amplifying nucleic acids.

An alternative means for generating the nucleic acid for the microarray is by synthesis of synthetic polynucleotides or oligonucleotides, e.g., using N-phosphonate or phosphoramidite chemistries. See Froehler et al., Nucl. Acid Res., Vol. 14, pp. 5399-5407 (1986); and McBride et al., Tetrahedron Lett., Vol. 24, pp. 245-248 (1983). Synthetic sequences are between about 15 bases and about 500 bases in length, more typically between about 20 bases and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid. See, e.g., Egholm et al., “PNA Hybridizes to Complementary Oligonucleotides Obeying the Watson-Crick Hydrogen-Bonding Rules”, Nature, Vol. 365, pp. 566-568 (1993); and U.S. Pat. No. 5,539,083.

In an alternative embodiment, the binding (hybridization) sites are made from plasmid or phage clones of genes, cDNAs (e.g., ESTS), or inserts therefrom. See Nguyen et al., “Differential Gene Expression in the Murine Thymus Assayed by Quantitative Hybridization of Arrayed cDNA Clones”, Genomics, Vol. 29, pp. 207-209 (1995). In yet another embodiment, the polynucleotide of the binding sites is RNA.

Attaching Nucleic Acids to the Solid Surface

The nucleic acid or analogue are attached to a solid support, which may be made from glass, plastic (e.g., polypropylene, nylon), polyacrylamide, nitrocellulose or other materials. A preferred method for attaching the nucleic acids to a surface is by printing on glass plates, as is described generally by Schena et al., “Quantitative Monitoring of Gene Expression Patterns with a Complementary DNA Microarray”, Science, Vol. 270, pp. 467-470 (1995). This method is especially useful for preparing microarrays of cDNA. See, also, DeRisi et al., “Use of a cDNA Microarray to Analyze Gene Expression Patterns in Human Cancer”, Nature Gen., Vol. 14, pp. 457460 (1996); Shalon et al., “A DNA Microarray System for Analyzing Complex DNA Samples Using Two-Color Fluorescent Probe Hybridization”, Genome Res., Vol. 6, pp. 639-645 (1996); and Schena et al., “Parallel Human Genome Analysis; Microarray-Based Expression of 1000 Genes”, Proc. Natl. Acad. Sci. USA, Vol. 93, pp. 10539-11286 (1995). Each of the aforementioned articles is incorporated by reference in its entirety for all purposes.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ, see Fodor et al., “Light-Directed Spatially Addressable Parallel Chemical Synthesis”, Science, Vol. 251, pp. 767-773 (1991); Pease et al., “Light-Directed Oligonucleotide Arrays for Rapid DNA Sequence Analysis”, Proc. Natl. Acad. Sci. USA, Vol. 91, pp. 5022-5026 (1994); Lockhart et al., “Expression Monitoring by Hybridization to High-Density Oligonucleotide Arrays”, Nature Biotech., Vol. 14, p. 1675 (1996); and U.S. Pat. Nos. 5,578,832; 5,556,752; and 5,510,270, each of which is incorporated by reference in its entirety for all purposes; or other methods for rapid synthesis and deposition of defined oligonucleotides. See Blanchard et al., “High-Density Oligonucleotide Arrays”, Biosensors Bioelectron., Vol. 11, pp. 687-690 (1996). When these methods are used, oligonucleotides (e.g., 25 mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking, see Maskos et al., Nuc. Acids Res., Vol. 20, pp. 1679-1684 (1992), may also be used. In principal, any type of array, for example, dot blots on a nylon hybridization membrane. See Sambrook et al., “Molecular Cloning—A Laboratory Manual”, 2^{nd} Edition, Vols. 1-3, Cold Spring Harbor Laboratory, Cold Spring Harbor, N.Y. (1989), which is incorporated in its entirety for all purposes, could be used, although, as will be recognized by those of skill in the art, very small arrays will be preferred because hybridization volumes will be smaller.

Generating Labeled Probes

Methods for preparing total and poly(A)⁺ RNA are well-known and are described generally in Sambrook et al., supra. In one embodiment, RNA is extracted from cells of the various types of interest in this invention using guanidinium thiocyanate lysis followed by CsCl centrifugation. See Chirgwin et al., Biochemistry, Vol. 18, pp. 5294-5299 (1979). Poly(A)⁺ RNA is selected by selection with oligo-dT cellulose. See Sambrook et al., supra. Cells of interest include wild-type cells, drug-exposed wild-type cells, cells with modified/perturbed cellular constituent(s), and drug-exposed cells with modified/perturbed cellular constituent(s).

Labeled cDNA is prepared from mRNA or alternatively directly from RNA by oligo dT-primed or random-primed reverse transcription, both of which are well-known in the art. See, e.g., Klug et al., Methods Enzymol., Vol. 152, pp. 316-325 (1987). Reverse transcription may be carried out in the presence of a dNTP conjugated to a detectable label, most preferably a fluorescently-labeled dNTP. Alternatively, isolated mRNA can be converted to labeled antisense RNA synthesized by in vitro transcription of double-stranded cDNA in the presence of labeled dNTPs. See Lockhart et al., “Expression Monitoring by Hybridization to High-Density Oligonucleotide Arrays”, Nature Biotech., Vol. 14, p. 1675 (1996), which is incorporated by reference in its entirety for all purposes. In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, Fluor X (Amersham) and others. See, e.g., Kricka, Nonisotopic DNA Probe Techniques, Academic Press, San Diego, Calif. (1992). It will be appreciated that pairs of fluorophores are chosen that have distinct emission spectra so that they can be easily distinguished.

In another embodiment, a label other than a fluorescent label is used. For example, a radioactive label or a pair of radioactive labels with distinct emission spectra, can be used. See Zhao et al., “High Density cDNA Filter Analysis: A Novel Approach for Large-Scale, Quantitative Analysis of Gene Expression”, Gene, Vol. 156, p. 207 (1995); and Pietu et al., “Novel Gene Transcripts Preferentially Expressed in Human Muscles Revealed by Quantitative Hybridization of a High Density cDNA Array”, Genome Res., Vol. 6, p. 492 (1996). However, because of scattering of radioactive particles, and the consequent requirement for widely-spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., ™II, LTI Inc.) at 42° C. for 60 minutes.

Hybridization to Microarrays

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls. See, e.g., Shalon et al., supra; and Chee et al., supra.

Optimal hybridization conditions will depend on the length (e.g., oligomer vs. polynucleotide >200 bases) and type (e.g., RNA, DNA and PNA) of labeled probe and immobilized polynucleotide or oligonucleotide. General parameters for specific (i.e., stringent) hybridization conditions for nucleic acids are described in Sambrook et al., supra; and Ausubel et al., Current Protocols in Molecular Biology, Greene Publishing and Wiley-Interscience, NY (1987), which is incorporated in its entirety for all purposes. When the cDNA microarrays of Schena et al. are used, typical hybridization conditions are hybridization in 5×SSC plus 0.2% SDS at 65° C. for 4 hours followed by washes at 25° C. in low-stringency wash buffer (1×SSC plus 0.2% SDS) followed by 10 minutes at 25° C. in high-stringency wash buffer (0.1×SSC plus 0.2% SDS). See Shena et al., Proc. Natl. Acad. Sci. USA, Vol. 93, p. 10614 (1996). Useful hybridization conditions are also provided in, e.g., Tijessen, Hybridization with Nucleic Acid Probes, Elsevier Science, Publishers B.V. and Kricka (1993); and “Nonisotopic DNA Probe Techniques”, Academic Press, San Diego, Calif. (1992).

Signal Detection and Data Analysis

When fluorescently-labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows specimen illumination at wavelengths specific to the fluorophores used and emissions from the fluorophore can be analyzed. In a preferred embodiment, the arrays are scanned with a laser fluorescent scanner with a computer controlled X-Y stage and a microscope objective. Sequential excitation of the fluorophore is achieved with a multi-line, mixed gas laser and the emitted light is split by wavelength and detected with a photomultiplier tube. Fluorescence laser scanning devices are described in Schena et al., Genome Res., Vol. 6, pp. 639-645 (1996) and in other references cited herein. Alternatively, the fiber-optic bundle described by Ferguson et al., Nature Biotechnol., Vol. 14, pp. 1681-1684 (1996), may be used to monitor mRNA abundance levels at a large number of sites simultaneously.

Signals are recorded and, in a preferred embodiment, analyzed by computer, e.g., using a 12-bit analog to digital board. In one embodiment the scanned image is despeckled using a graphics program (e.g., Hijaak Graphics Suite) and then analyzed using an image gridding program that creates a spreadsheet of the average hybridization at each wavelength at each site.

The Agilent Technologies GENEARRAY™ scanner is a bench-top, 488 nM argon-ion laser-based analysis instrument. The laser can be focused to a spot size of less than 4 microns. This precision allows for the scanning of probe arrays with probe cells as small as 20 microns. The laser beam focuses onto the probe array, exciting the fluorescent-labeled nucleotides. It then and then scans using the selected filter for the dye used in the assay. Scanning in the orthogonal coordinate is achieved by moving the probe array. The laser radiation is absorbed by the dye molecules incorporated into the hybridized sample and causes them to emit fluorescence radiation. This fluorescent light is collimated by a lens and passes through a filter for wavelength selection. The light is then focused by a second lens onto an aperture for depth discrimination and then detected by a highly sensitive photo multiplier tube (PMT). The output current of the PMT is converted into a voltage read by an analog to digital converter and the processed data is passed back to the computer as the fluorescent intensity level of the sample point, or picture element (pixel) currently being scanned. The computer displays the data as an image, as the scan progresses. In addition, the fluorescent intensity level of all samples, representing the expression profile of the sample, is recorded in computer readable format.

If necessary, an experimentally determined correction for “cross talk” (or overlap) between the channels for the two fluors may be made. For any particular hybridization site on the transcript array, a ratio of the emission of the two fluorophores may be calculated. The ratio is independent of the absolute expression level of the cognate gene, but may be useful for genes whose expression is significantly modulated by drug administration, gene deletion, or any other tested event.

Preferably, in addition to identifying a perturbation as positive or negative, it is advantageous to determine the magnitude of the perturbation. This can be carried out by methods that will be readily apparent to those of skill in the art.

Other Methods of Transcriptional State Measurement

The transcriptional state of a cell may be measured by other gene expression technologies known in the art. Several such technologies produce pools of restriction fragments of limited complexity for electrophoretic analysis, such as methods combining double restriction enzyme digestion with phasing primers, see, e.g., European Patent application 0 534858 A1, filed Sep. 24, 1992, by Zabeau et al., or methods selecting restriction fragments with sites closest to a defined mRNA end. See, e.g., Prashar et al., Proc. Natl. Acad. Sci. USA, Vol. 93, pp. 659-663 (1996). Other methods statistically sample cDNA pools, such as by sequencing sufficient bases (e.g., 20-50 bases) in each of multiple cDNAs to identify each cDNA, or by sequencing short tags (e.g., 9-10 bases) which are generated at known positions relative to a defined mRNA end, see, e.g., Velculescu, Science, Vol. 270, pp. 484487 (1995), pathway pattern.

Measurement of Other Aspects

In various embodiments of the present invention, aspects of the biological state other than the transcriptional state, such as the translational state, the activity state or mixed aspects can be measured in order to obtain drug and pathway responses. Details of these embodiments are described in this section.

Translational State Measurements

Expression of the protein encoded by the gene(s) can be detected by a probe which is detectably-labeled, or which can be subsequently-labeled. Generally, the probe is an antibody that recognizes the expressed protein.

As used herein, the term “antibody” includes, but is not limited to, polyclonal antibodies, monoclonal antibodies, humanized or chimeric antibodies and biologically functional antibody fragments sufficient for binding of the antibody fragment to the protein.

For the production of antibodies to a protein encoded by one of the disclosed genes, various host animals may be immunized by injection with the polypeptide, or a portion thereof. Such host animals may include, but are not limited to, rabbits, mice and rats, to name but a few. Various adjuvants may be used to increase the immunological response, depending on the host species, including, but not limited to, Freund's (complete and incomplete); mineral gels, such as aluminum hydroxide; surface active substances, such as lysolecithin, pluronic polyols, polyanions, peptides, oil emulsions, keyhole limpet hemocyanin, dinitrophenol; and potentially useful human adjuvants, such as BCG and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler et al., Nature, Vol. 256, pp. 495-497 (1975); and U.S. Pat. No. 4,376,110. The human B-cell hybridoma technique of Kosbor et al., Immunol. Today, Vol. 4, p. 72 (1983); Cole et al., Proc. Natl. Acad. Sci. USA, Vol. 80, pp. 2026-2030 (1983); and the EBV-hybridoma technique, Cole et al., Monoclonal Antibodies and Cancer Ther., Alan R. Liss, Inc., pp. 77-96 (1985). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies”, see Morrison et al., Proc. Natl. Acad. Sci. USA, Vol. 81, pp. 6851-6855 (1984); Neuberger et al., Nature, Vol. 312, pp. 604-608 (1984); and Takeda et al., Nature, Vol. 314, pp. 452-454 (1985), by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived form a murine mAb and a human immunoglobulin constant region.

Alternatively, techniques described for the production of single-chain antibodies; see U.S. Pat. No. 4,946,778; Bird, Science, Vol. 242, pp. 423-426 (1988); Huston et al., Proc. Natl. Acad. Sci. USA, Vol. 85, pp. 5879-5883 (1988); and Ward et al., Nature, Vol. 334, pp. 544-546 (1989); can be adapted to produce differentially-expressed gene single-chain antibodies. Single-chain antibodies are formed by linking the heavy and light chain fragments of the Fv region via an amino acid bridge, resulting in a single-chain polypeptide.

More preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments, which recognize specific epitopes, may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed; see Huse et al., Science, Vol. 246, pp. 1275-1281 (1989); to allow rapid and easy identification of monoclonal Fab fragments with the desired specificity.

The extent to which the known proteins are expressed in the sample is then determined by immunoassay methods that utilize the antibodies described above. Such immunoassay methods include, but are not limited to, dot blotting, western blotting, competitive and non-competitive protein-binding assays, enzyme-linked immunosorbant assays (ELISA), immunohistochemistry, fluorescence activated cell sorting (FACS) and others commonly-used and widely-described in scientific and patent literature, and many employed commercially.

Particularly preferred, for ease of detection, is the sandwich ELISA, of which a number of variations exist, all of which are intended to be encompassed by the present invention. For example, in a typical forward assay, unlabeled antibody is immobilized on a solid substrate and the sample to be tested brought into contact with the bound molecule after a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well-known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody must be an antibody that is specific for the protein expressed by the gene of interest.

The most commonly used reporter molecules in this type of assay are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, β-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of protein which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Measurement of the translational state may also be performed according to several additional methods. For example, whole genome monitoring of protein (i.e., the “proteome”, see Goffeau et al., supra) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. Methods for making monoclonal antibodies are well-known. See, e.g., Harlow et al., Antibodies: A Laboratory Manual, Cold Spring Harbor, N.Y. (1988), which is incorporated in its entirety for all purposes. In a preferred embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., Gel Electrophoresis of Proteins: A Practical Approach, IRL Press, NY (1990); Shevchenko et al., Proc. Natl Acad. Sci. USA, Vol. 93, pp. 1440-1445 (1996); Sagliocco et al., Yeast, Vol. 12, pp. 1519-1533 (1996); and Lander, Science, Vol. 274, pp. 536-539 (1996). The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

Embodiments Based on Other Aspects of the Biological State

Although monitoring cellular constituents other than mRNA abundances currently presents certain technical difficulties not encountered in monitoring mRNAs, it will be apparent to those of skill in the art that the use of methods of this invention that the activities of proteins relevant to the characterization of cell function can be measured, embodiments of this invention can be based on such measurements. Activity measurements can be performed by any functional, biochemical, or physical means appropriate to the particular activity being characterized. Where the activity involves a chemical transformation, the cellular protein can be contacted with the natural substrates, and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In alternative and non-limiting embodiments, response data may be formed of mixed aspects of the biological state of a cell. Response data can be constructed from, e.g., changes in certain mRNA abundances, changes in certain protein abundances, and changes in certain protein activities.

Utilization of SNPs for Predication of Response

SNPs

Sequence variation in the human genome consists primarily of SNPs with the remainder of the sequence variations being short tandem repeats (including micro-satellites), long tandem repeats (mini-satellite) and other insertions and deletions. A SNP is a position at which two alternative bases occur at appreciable frequency, such as >1%, in the human population. A SNP is said to be “allelic” in that due to the existence of the polymorphism, some members of a species may have the unmutated sequence, such as the original “allele”, whereas other members may have a mutated sequence, i.e., the variant or mutant allele. In the simplest case, only one mutated sequence may exist, and the polymorphism is said to be di-allelic. The occurrence of alternative mutations can give rise to tri-allelic polymorphisms, etc. SNPs are widespread throughout the genome and SNPs that alter the function of a gene may be direct contributors to phenotypic variation. Due to their prevalence and widespread nature, SNPs have potential to be important tools for locating genes that are involved in human disease conditions. See, e.g., Wang et al., Science, Vol. 280, pp. 1077-1082 (1998), which discloses a pilot study in which 2,227 SNPs were mapped over a 2.3 megabase region of DNA.

An association between a SNPs and a particular phenotype does not indicate or require that the SNP is causative of the phenotype. Instead, such an association may indicate only that the SNP is located near the site on the genome where the determining factors for the phenotype exist and therefore is more likely to be found in association with these determining factors and thus with the phenotype of interest. Thus, a SNP may be in LD with the ‘true’ functional variant. LD, also known as allelic association exists when alleles at two distinct locations of the genome are more highly associated than expected. Thus a SNP may serve as a marker that has value by virtue of its proximity to a mutation that causes a particular phenotype. SNPs that are associated with disease may also have a direct effect on the function of the gene in which they are located. A sequence variant may result in an amino add change or may alter exon-intron splicing, thereby directly modifying the relevant protein, or it may exist in a regulatory region, altering the cycle of expression or the stability of the messenger RNA (mRNA). See Nowotnym, Curr. Opin. Neurobiol., Vol. 11, pp. 637-641 (2001).

The role that a common genomic variant might play in susceptibility to disease is best exemplified by the role that the APOE ε4 allele plays in AD. The ε4 allele is highly associated with the presence of AD and with earlier age of onset of disease. It is a robust association seen in many populations studied. See St. George-Hyslop et al., Biol. Psychiatry, Vol. 47, pp. 183-199 (2000). Polymorphic variation has also been implicated in stroke and cardiovascular disease, see Wu et al., Am. J. Cardiol., Vol. 87, pp. 1361-1366 (2001); and in multiple sclerosis, see Oksenberg et al., J. Neuroimmuol., Vol. 113, pp. 171-184 (2001).

It is increasingly clear that the risk of developing many common disorders and the individuals response to medication and the metabolism of medications used to treat these conditions are substantially influenced by underlying genomic variations, although the effects of any one variant might be small.

Therefore, an association between a SNP and a clinical phenotype suggests: 1) the SNP is functionally responsible for the phenotype; or 2) there are other mutations near the location of the SNP on the genome that cause the phenotype. The second possibility is based on the biology of inheritance. Large pieces of DNA are inherited and markers in close proximity to each other may not have been recombined in individuals that are unrelated for many generations, i.e., the markers are in LD.

The use of polymorphisms as genetic linkage markers is thus of critical importance in locating, identifying and characterizing the genes which are responsible for specific traits. In particular, such mapping techniques allow for the identification of genes responsible for a variety of disease or disorder-related traits including the response of the disorder to various treatments.

Identification and Characterization of SNPs

Many different techniques can be used to identify and characterize SNPs, including single-strand conformation polymorphism analysis, heteroduplex analysis by denaturing high-performance liquid chromatography (DHPLC), direct DNA sequencing and computational methods. See Shi, Clin. Chem., Vol. 47, pp. 164-172 (2001). Thanks to the wealth of sequence information in public databases, computational tools can be used to identify SNPs in silico by aligning independently submitted sequences for a given gene (either cDNA or genomic sequences). Comparison of SNPs obtained experimentally and by in silico methods showed that 55% of candidate SNPs found by SNPFinder(http://lpgws.nci.nih.gov:82/perl/snp/snp_cgi.pl) have also been discovered experimentally. See Cox et al., Hum. Mutal., Vol. 17, pp. 141-150 (2001). However, these in silico methods could only find 27% of true SNPs.

The most common SNP typing methods currently include hybridization, primer extension and cleavage methods. Each of these methods must be connected to an appropriate detection system. Detection technologies include fluorescent polarization, (see Chan et al., Genome Res., Vol. 9, pp. 492-499 (1999)), luminometric detection of pyrophosphate release (pyrosequencing), (see Ahmadiian et al., Anal. Biochem., Vol. 280, pp. 103-110 (2000)), fluorescence resonance energy transfer (FRET)-based cleavage assays, DHPLC, and mass spectrometry. See Shi, Clin. Chem., Vol. 47, pp. 164-172 (2001); and U.S. Pat. No. 6,300,076 B1. Other methods of detecting and characterizing SNPs are those disclosed in U.S. Pat. Nos. 6,297,018 B1 and 6,300,063 B1. The disclosures of the above references are incorporated herein by reference in their entirety.

In a particularly preferred embodiment the detection of the polymorphism can be accomplished by means of so called INVADER™ technology (available from Third Wave Technologies Inc.). In this assay, a specific upstream “invader” oligonucleotide and a partially overlapping downstream probe together form a specific structure when bound to complementary DNA template. This structure is recognized and cut at a specific site by the Cleavase enzyme, and this results in the release of the 5′ flap of the probe oligonucleotide. This fragment then serves as the “invader” oligonucleotide with respect to synthetic secondary targets and secondary fluorescently-labeled signal probes contained in the reaction mixture. This results in specific cleavage of the secondary signal probes by the Cleavase enzyme. Fluorescence signal is generated when this secondary probe, labeled with dye molecules capable of fluorescence resonance energy transfer, is cleaved. Cleavases have stringent requirements relative to the structure formed by the overlapping DNA sequences or flaps and can, therefore, be used to specifically detect single base pair mismatches immediately upstream of the cleavage site on the downstream DNA strand. See Ryan et al., Molecular Diagnosis, Vol. 4, No 2, pp. 135-144 (1999); and Lyamichev et al., Nat. Biotechnol., Vol. 17, pp. 292-296 (1999); see also U.S. Pat. Nos. 5,846,717 and 6,001,567 (the disclosures of which are incorporated herein by reference in their entirety).

In some embodiments, a composition contains two or more differently labeled genotyping oligonucleotides for simultaneously probing the identity of nucleotides at two or more polymorphic sites. It is also contemplated that primer compositions may contain two or more sets of allele-specific primer pairs to allow simultaneous targeting and amplification of two or more regions containing a polymorphic site.

IL-1β genotyping oligonucleotides of the invention may also be immobilized on or synthesized on a solid surface such as a microchip, bead or glass slide (see, e.g., WO 98/20020 and WO 98/20019). Such immobilized genotyping oligonucleotides may be used in a variety of polymorphism detection assays, including but not limited to probe hybridization and polymerase extension assays. Immobilized IL-1β genotyping oligonucleotides of the invention may comprise an ordered array of oligonucleotides designed to rapidly screen a DNA sample for polymorphisms in multiple genes at the same time.

An allele-specific oligonucleotide primer of the invention has a 3′ terminal nucleotide, or preferably a 3′ penultimate nucleotide, that is complementary to only one nucleotide of a particular SNP, thereby acting as a primer for polymerase-mediated extension only if the allele containing that nucleotide is present. Allele-specific oligonucleotide primers hybridizing to either the coding or noncoding strand are contemplated by the invention. An ASO primer for detecting IL-1β gene polymorphisms could be developed using techniques known to those of skill in the art.

Other genotyping oligonucleotides of the invention hybridize to a target region located one to several nucleotides downstream of one of the novel polymorphic sites identified herein. Such oligonucleotides are useful in polymerase-mediated primer extension methods for detecting one of the novel polymorphisms described herein and therefore such genotyping oligonucleotides are referred to herein as “primer-extension oligonucleotides”. In a preferred embodiment, the 3′-terminus of a primer-extension oligonucleotide is a deoxynucleotide complementary to the nucleotide located immediately adjacent to the polymorphic site.

In another embodiment, the invention provides a kit comprising at least two genotyping oligonucleotides packaged in separate containers. The kit may also contain other components, such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as PCR. The above described oligonucleotide compositions and kits are useful in methods for genotyping and/or haplotyping the IL-1β gene in an individual.

One embodiment of the genotyping method involves isolating from the individual a nucleic acid mixture comprising the two copies of the IL-1β gene, or a fragment thereof, that are present in the individual, and determining the identity of the nucleotide pair at one or more of the polymorphic sites in the two copies to assign a IL-1β genotype to the individual. As will be readily understood by the skilled artisan, the two “copies” of a gene in an individual may be the same allele or may be different alleles. In a particularly preferred embodiment, the genotyping method comprises determining the identity of the nucleotide pair at each polymorphic site.

Typically, the nucleic acid mixture or protein is isolated from a biological sample taken from the individual, such as a blood sample or tissue sample. Suitable tissue samples include whole blood, serum, semen, saliva, tears, urine, fecal material, sweat, buccal smears, skin and biopsies of specific organ tissues, such as muscle or nerve tissue and hair. The nucleic acid mixture may be comprised of genomic DNA, mRNA or cDNA and, in the latter two cases, the biological sample must be obtained from an organ in which the IL-1β gene is expressed. Furthermore it will be understood by the skilled artisan that mRNA or cDNA preparations would not be used to detect polymorphisms located in introns, in 5′ and 3′ non-transcribed regions or in promoter regions. If an IL-1β gene fragment is isolated, it must contain the polymorphic site(s) to be genotyped.

One embodiment of the haplotyping method comprises isolating from the individual a nucleic acid molecule containing only one of the two copies of the IL-1β gene, or a fragment thereof, that is present in the individual and determining in that copy the identity of the nucleotide at one or more of the polymorphic sites in that copy to assign a IL-1β haplotype to the individual. The nucleic acid may be isolated using any method capable of separating the two copies of the IL-1β gene or fragment, including but not limited to, one of the methods described above for preparing IL-1β isogenes, with targeted in vivo cloning being the preferred approach.

As will be readily appreciated by those skilled in the art, any individual clone will only provide haplotype information on one of the two IL-1β gene copies present in an individual. If haplotype information is desired for the individuals other copy, additional IL-1β clones will need to be examined. Typically, at least five clones should be examined to have more than a 90% probability of haplotyping both copies of the IL-1β gene in an individual. In a particularly preferred embodiment, the nucleotide at each of polymorphic site is identified.

In a preferred embodiment, a IL-1β haplotype pair is determined for an individual by identifying the phased sequence of nucleotides at one or more of the polymorphic sites in each copy of the IL-1β gene that is present in the individual. In a particularly preferred embodiment, the haplotyping method comprises identifying the phased sequence of nucleotides at each polymorphic site in each copy of the IL-1β gene. When haplotyping both copies of the gene, the identifying step is preferably performed with each copy of the gene being placed in separate containers. However, it is also envisioned that if the two copies are labeled with different tags, or are otherwise separately distinguishable or identifiable, it could be possible in some cases to perform the method in the same container. For example, if first and second copies of the gene are labeled with different first and second fluorescent dyes, respectively, and an allele-specific oligonucleotide labeled with yet a third different fluorescent dye is used to assay the polymorphic site(s), then detecting a combination of the first and third dyes would identify the polymorphism in the first gene copy while detecting a combination of the second and third dyes would identify the polymorphism in the second gene copy.

In both the genotyping and haplotyping methods, the identity of a nucleotide (or nucleotide pair) at a polymorphic site(s) may be determined by amplifying a target region(s) containing the polymorphic site(s) directly from one or both copies of the IL-1β gene, or fragment thereof, and the sequence of the amplified region(s) determined by conventional methods. It will be readily appreciated by the skilled artisan that the same nucleotide will be detected twice at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a SNP is known to be guanine and cytosine in a reference population, a site may be positively determined to be either guanine or cytosine for all individual homozygous at that site, or both guanine and cytosine, if the individual is heterozygous at that site. Alternatively, the site may be negatively determined to be not guanine (and thus cytosine/cytosine) or not cytosine (and thus guanine/guanine).

In addition, the identity of the allele(s) present at any of the novel polymorphic sites described herein may be indirectly determined by genotyping a polymorphic site not disclosed herein that is in linkage disequilibrium with the polymorphic site that is of interest. Two sites are said to be in linkage disequilibrium if the presence of a particular variant at one site enhances the predictability of another variant at the second site. See Stevens, Mol. Diag., Vol. 4, pp. 309-317 (1999). Polymorphic sites in linkage disequilibrium with the presently disclosed polymorphic sites may be located in regions of the gene or in other genomic regions not examined herein. Genotyping of a polymorphic site in linkage disequilibrium with the novel polymorphic sites described herein may be performed by, but is not limited to, any of the above-mentioned methods for detecting the identity of the allele at a polymorphic site.

The target region(s) may be amplified using any oligonucleotide-directed amplification method, including but not limited to polymerase chain reaction (PCR) (U.S. Pat. No. 4,965,188), ligase chain reaction (see Barany et al., Proc. Natl. Acad. Sci. USA, Vol. 88, pp. 189-193 (1991); and WO 90/01069), and oligonucleotide ligation assay. See Landegren et al., Science, Vol. 241, pp. 1077-1080 (1988). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20-25 nucleotides long. The exact length of the oligonucleotide will depend on many factors that are routinely considered and practiced by the skilled artisan.

Other known nucleic acid amplification procedures may be used to amplify the target region including transcription-based amplification systems (see U.S. Pat. Nos. 5,130,238 and 5,169,766; EP 329,822; and WO 89/06700) and isothermal methods. See Walker et al., Proc. Natl. Acad. Sci. USA, Vol. 89, pp. 392-396 (1992).

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within 5° C. and more preferably within 2° C., of each other when hybridizing to each of the polymorphic sites being detected.

Hybridization of an allele-specific oligonucleotide to a target polynucleotide may be performed with both entities in solution or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes and beads. The solid support may be treated, coated or derivatized to facilitate the immobilization of the allele-specific oligonucleotide or target nucleic acid.

The genotype or haplotype for the IL-1β gene of an individual may also be determined by hybridization of a nucleic sample containing one or both copies of the gene to nucleic acid arrays and subarrays, such as described in WO 95/11995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (see Winter et al., Proc. Natl. Acad. Sci. USA, Vol. 82, p. 7575 (1985); and Meyers et al., Science, Vol. 230, p. 1242 (1985)) and proteins which recognize nucleotide mismatches, such as the E. coli mutS protein. See Modrich, Ann. Rev. Genet., Vol. 25, pp. 229-253 (1991). Alternatively, variant alleles can be identified by single-strand conformation polymorphism (SSCP) analysis (see Orita et al., Genomics, Vol. 5, pp. 874-879 (1989); and Humphries et al., Molecular Diagnosis of Genetic Diseases, Elles, Ed., pp. 321-340 (1996)) or denaturing gradient gel electrophoresis. See Wartell et at., Nucl. Acids Res., Vol. 18, pp. 2699-2706 (1990); and Sheffield et al., Proc. Natl. Acad. Sci. USA, Vol. 86, pp. 232-236 (1989).

A polymerase-mediated primer extension method may also be used to identify the polymorphism(s). Several such methods have been described in the patent and scientific literature and include the “Genetic Bit Analysis” method (WO 92/15712) and the ligase/polymerase mediated genetic bit analysis (see U.S. Pat. No. 5,679,524). Related methods are disclosed in WO 91/02087, WO 90/09455, WO 95/17676, U.S. Pat. Nos. 5,302,509 and 5,945,283. Extended primers containing a polymorphism may be detected by mass spectrometry. See U.S. Pat. No. 5,605,798. Another primer extension method is allele-specific PCR. See Ruafio et al., Nucl. Acids Res., Vol. 17, p. 8392 (1989); Ruaflo et al., Nucl. Acids Res., Vol. 19, pp. 6877-6882 (1991); WO 93/22456; and Turki et al., J. Clin. Invest., Vol. 95, pp. 1635-1641 (1995). In addition, multiple polymorphic sites may be investigated by simultaneously amplifying multiple regions of the nucleic acid using sets of allele-specific primers. See Wallace et al. (WO 89/10414).

In a preferred embodiment, the haplotype frequency data for each ethnogeographic group is examined to determine whether it is consistent with HWE. HWE (see Hartl et al., Principles of Population Genomics, Sinauer Associates, 3^{rd} Edition, Sunderland, Mass. (1997), postulates that the frequency of finding the haplotype pair H₁/H₂ is equal to P_{H-W} (H₁/H₂)=2p(H₁) p (H₂) if H₁≠H₂ and P_{H-W} (H₁/H₂)=p (H₁) p (H₂) if H₁=H₂. A statistically significant difference between the observed and expected haplotype frequencies could be due to one or more factors including significant inbreeding in the population group, strong selective pressure on the gene, sampling bias and/or errors in the genotyping process. If large deviations from HWE are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method, such as, e.g., CLASPER System™ technology (U.S. Pat. No. 5,866,404), SMD or allele-specific long-range PCR. See Michalotos-Beloin et al., Nucl. Acids Res., Vol. 24, pp. 4841-4843 (1996).

In one embodiment of this method for predicting an IL-1β haplotype pair, the assigning step involves performing the following analysis. First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair. In rare cases, either no haplotype in the reference population are consistent with the possible haplotype pairs, or alternatively, multiple reference haplotype pairs are consistent with the possible haplotype pairs. In such cases, the individual is preferably haplotyped using a direct molecular haplotyping method such as, for example, CLASPER System™ technology (see U.S. Pat. No. 5,866,404), SMD or allele-specific long-range PCR. See Michalotos-Beloin et al., supra.

Methods of Modifying the Abundance or Activity of mRNA

In various embodiments of this invention altering or modifying the abundance or activity of expressed mRNA produces clinically beneficial effects. Methods of modifying RNA abundance and activities currently fall within four classes; ribozymes, antisense species, double-stranded RNA and RNA aptamers. See Good et al., Gene Ther., Vol. 4, pp. 45-54 (1997). Controllable application or exposure of a cell to these entities permits controllable perturbation of RNA abundance including mRNA abundance and activity, including its translation into active or detectable gene expression products, i.e., proteins.

Ribozymes

Ribozymes are RNA molecules that specifically cleave other single-stranded RNA in a manner similar to DNA restriction endonucleases. Ribozymes are capable of catalyzing RNA cleavage reactions. See Cech, Science, Vol. 236, pp. 1532-1539 (1987); PCT International Publication WO 90/11364 (1990); and Sarver et al., Science, Vol. 247, pp. 1222-1225 (1990). By modifying the nucleotide sequences encoding the RNAs, ribozymes can be synthesized to recognize specific nucleotide sequences in a molecule and cleave it. See, e.g., in Cech, Amer. Med. Assn., Vol. 260, pp. 3030 (1988). Accordingly, only mRNAs with specific sequences are cleaved and inactivated.

Two basic types of ribozymes include the “hammerhead”-type as described, e.g., in Rossie et al., Pharmacol. Ther., Vol. 50, pp. 245-254 (1991); and the “hairpin” ribozyme as described, e.g., in Hampel et al., Nucl. Acids Res., Vol. 18, pp. 299-304 (1999) and U.S. Pat. No. 5,254,678. Hairpin and hammerhead RNA ribozymes can be designed to specifically cleave a particular target mRNA. Rules have been established for the design of short RNA molecules with ribozyme activity, which are capable of cleaving other RNA molecules in a highly sequence specific way and can be targeted to virtually all kinds of RNA. See Haseloff et al., Nature, Vol. 334, pp. 585-591 (1988); Koizumi et al., FEBS Lett., Vol. 228, pp. 228-230 (1988); and Koizumi et al., FEBS Lett., Vol. 239, pp. 285-288 (1988).

Ribozyme methods involve exposing a cell to, inducing expression in a cell, etc. of such small RNA ribozyme molecules. See Grassi et al., Ann. Med., Vol. 28, pp. 499-510 (1996); Gibson, Cancer and Metastasis Rev., Vol. 15, pp. 287-299 (1996). Intracellular expression of hammerhead and hairpin ribozymes targeted to mRNA corresponding to at least one of the disclosed genes can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell. See Coffen et al., “Ribozyme Mediated Destruction of RNA In Vivo”, EMBO J., Vol. 8, pp. 3861-3866 (1989). In particular, a ribozyme coding DNA sequence, designed according to the previous rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. For saturating use, a highly and constituently active promoter can be used. tDNA genes (i.e., genes encoding tRNAs) are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be modified or perturbed.

Ribozyme sequences can be modified in essentially the same manner as described for antisense nucleotides, e.g., the ribozyme sequence can comprise a modified base moiety.

Antisense Molecules

In another embodiment, activity of a target RNA (preferable mRNA) species, specifically its rate of translation, can be controllably inhibited by the controllable application of antisense nucleic acids. Application at high levels results in a saturating inhibition. An “antisense” nucleic acid as used herein refers to a nucleic acid capable of hybridizing to a sequence-specific (e.g., non-poly A) portion of the target RNA, for example, its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably oligonucleotides (ranging from 6 oligonucleotides to about 200 oligonucleotides). In specific aspects, the oligonucleotide is at least 10 nucleotides, at least 15 nucleotides, at least 100 nucleotides or at least 200 nucleotides. The oligonucleotides can be DNA or RNA or chimeric mixtures or derivatives or modified versions thereof, single-stranded or double-stranded. The oligonucleotide can be modified at the base moiety, sugar moiety or phosphate backbone. The oligonucleotide may include other appending groups, such as peptides, or agents facilitating transport across the cell membrane (see, e.g., Letsinger et al., Proc. Natl. Acad. Sci. USA, Vol. 86, pp. 6553-6556 (1989); Lemaitre et al., Proc. Natl. Acad. Sci. USA, Vol. 84, pp. 648-652 (1987); and PCT Publication No. WO 88/09810 (1988)), hybridization-triggered cleavage agents (see, e.g., Krol et al., Biotechnol. Tech., Vol. 6, pp. 958-976 (1988)) or intercalating agents. See, e.g., Zon, Pharmacol. Res., Vol. 5, pp. 539-549 (1988).

In a preferred aspect of the invention, an antisense oligonucleotide is provided, preferably as single-stranded DNA. The oligonucleotide may be modified at any position on its structure with constituents generally known in the art.

Typical antisense approaches involve the preparation of oligonucleotides, either DNA or RNA that are complementary to the encoded mRNA of the gene. The antisense oligonucleotides will hybridize to the encoded mRNA of the gene and prevent translation. The capacity of the antisense nucleotide sequence to hybridize with the desired gene will depend on the degree of complementarity and the length of the antisense nucleotide sequence. Typically, as the length of the hybridizing nucleic acid increases, the more base mismatches with an RNA it may contain and still form a stable duplex or triplex. One skilled in the art can determine a tolerable degree of mismatch by use of conventional procedures to determine the melting point of the hybridized complexes.

Antisense oligonucleotides are preferably designed to be complementary to the 5′ end of the mRNA, e.g., the untranslated sequence up to, and including, the regions complementary to the mRNA initiation site, i.e., AUG. However, oligonucleotide sequences that are complementary to the 3′ untranslated sequence of mRNA have also been shown to be effective at inhibiting translation of mRNAs. See, e.g., in Wagner, Nature, Vol. 372, p. 333 (1994). While antisense oligonucleotides can be designed to be complementary to the mRNA coding regions, such oligonucleotides are less efficient inhibitors of translation.

The antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w and 2,6-diaminopurine.

In another embodiment, the oligonucleotide comprises at least one modified sugar moiety selected from the group including, but not limited to, arabinose, 2-fluoroarabinose, xylulose and hexose.

In yet another embodiment, the oligonucleotide comprises at least one modified phosphate backbone selected from the group consisting of: a phosphorothioate, a phosphorodithioate, a phosphoramidothioate, a phosphoramidate, a phosphordiamidate, a methylphosphonate, an alkyl phosphotriester and a formacetal or analog thereof.

In yet another embodiment, the oligonucleotide is a 2-a-anomeric oligonucleotide. An a-anomeric oligonucleotide forms specific double-stranded hybrids with complementary RNA in which, contrary to the usual B-units, the strands run parallel to each other. See Gautier et al., Nucl. Acids Res., Vol. 15, pp. 6625-6641 (1987).

The oligonucleotide may be conjugated to another molecule, e.g., a peptide, hybridization triggered cross-linking agent, transport agent, hybridization-triggered cleavage agent, etc.

The antisense nucleic acids of the invention comprise a sequence complementary to at least a portion of a target RNA species. However, absolute complementarity, although preferred, is not required. A sequence “complementary to at least a portion of an RNA”, as referred to herein, means a sequence having sufficient complementarity to be able to hybridize with the RNA, forming a stable duplex; in the case of double-stranded antisense nucleic acids, a single-strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a target RNA it may contain and still form a stable duplex (or triplex, as the case may be). One skilled in the art can ascertain a tolerable degree of mismatch by use of standard procedures to determine the melting point of the hybridized complex. The amount of antisense nucleic acid that will be effective in the inhibiting translation of the target RNA can be determined by standard assay techniques.

Oligonucleotides of the invention may be synthesized by standard methods known in the art, e.g., by use of an automated DNA synthesizer, such as are commercially available from Biosearch, Applied Biosystems, etc. As examples, phosphorothioate oligonucleotides may be synthesized by the method of Stein et al., Nucl. Acids Res., Vol. 16, p. 3209 (1988), methylphosphonate oligonucleotides can be prepared by use of controlled pore glass polymer supports (see Sarin et al., Proc. Natl. Acad. Sci. USA, Vol. 85, pp. 7448-7451 (1988)), etc. In another embodiment, the oligonucleotide is a 2′-0-methylribonucleotide (see Inoue et al., Nucl. Acids Res., Vol. 15, pp. 6131-6148 (1987)) or a chimeric RNA-DNA analog. See Inoue et al., FEBS Lett., Vol. 215, pp. 327-330 (1987).

The synthesized antisense oligonucleotides can then be administered to a cell in a controlled or saturating manner. For example, the antisense oligonucleotides can be placed in the growth environment of the cell at controlled levels where they may be taken up by the cell. The uptake of the antisense oligonucleotides can be assisted by use of methods well-known in the art.

When introduced into a host cell, antisense nucleotide sequences specifically hybridize with the cellular mRNA and/or genomic DNA corresponding to the gene(s) so as to inhibit expression of the encoded protein, e.g., by inhibiting transcription and/or translation within the cell.

The isolated nucleic acid molecule comprising the antisense nucleotide sequence can be delivered, e.g., as an expression vector, which when transcribed in the cell, produces RNA which is complementary to at least a unique portion of the encoded mRNA of the gene(s). Alternatively, the isolated nucleic acid molecule comprising the antisense nucleotide sequence is an oligonucleotide probe which is prepared ex vivo and, which when introduced into the cell, results in inhibiting expression of the encoded protein by hybridizing with the mRNA and/or genomic sequences of the gene(s).

Preferably, the oligonucleotide contains artificial internucleotide linkages, which render the antisense molecule resistant to exonucleases and endonucleases, and thus are stable in the cell. Examples of modified nucleic acid molecules for use as antisense nucleotide sequences are phosphoramidate, phosporothioate and methylphosphonate analogs of DNA. See, e.g., U.S. Pat. Nos. 5,176,996; 5,264,564; and 5,256,775. General approaches to preparing oligomers useful in antisense therapy. See, e.g., Van der Krol., Biotechnol. Tech., Vol. 6, pp. 958-976 (1988); and Stein et al., Cancer Res., Vol. 48, pp. 2659-2668 (1988).

Antisense Molecules Expressed Intracellularly

As discussed above, antisense nucleotides can be delivered to cells which express the IL1β gene in vivo by various techniques. However, with it may be difficult to attain intracellular concentrations sufficient to inhibit translation of endogenous mRNA. Accordingly, in an alternative embodiment, the nucleic acid comprising an antisense nucleotide sequence is placed under the transcriptional control of a promoter, i.e., a DNA sequence which is required to initiate transcription of the specific genes, to form an expression construct. The antisense nucleic acids of the invention are controllably expressed intracellularly by transcription from an exogenous sequence. If the expression is controlled to be at a high level, a saturating perturbation or modification results. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequences encoding the antisense RNAs can be by any promoter known in the art to act in a cell of interest. Such promoters can be inducible or constitutive. Most preferably, promoters are controllable or inducible by the administration of an exogenous moiety in order to achieve controlled expression of the antisense oligonucleotide. Such controllable promoters include the Tet promoter. Other usable promoters for mammalian cells include, but are not limited to, the SV40 early promoter region (see Bernoist and Chambon, Nature, Vol. 290, pp. 304-310 (1981)), the promoter contained in the 3′ long terminal repeat of Rous sarcoma virus (see Yamamoto et al., Cell, Vol. 22, pp. 787-797 (1980)), the herpes thymidine kinase promoter (see Wagner et al., Proc. Natl. Acad. Sci. USA, Vol. 78, pp. 1441-1445 (1981)), the regulatory sequences of the metallothionein gene (see Brinster et al., Nature, Vol. 296, pp. 3942 (1982)), etc.

Therefore, antisense nucleic acids can be routinely designed to target virtually any mRNA sequence, and a cell can be routinely transformed with or exposed to nucleic acids coding for such antisense sequences such that an effective and controllable or saturating amount of the antisense nucleic acid is expressed. Accordingly the translation of virtually any RNA species in a cell can be modified or perturbed.

Double-Stranded RNA

Double-stranded RNA, i.e., sense-antisense RNA, corresponding to at least one of the disclosed genes, can also be utilized to interfere with expression of at least one of the disclosed genes. Interference with the function and expression of endogenous genes by double-stranded RNA has been shown in various organisms, such as C. elegans. See, e.g., Fire et al., Nature, Vol. 391, pp. 806-811 (1998).

RNA Aptamers

Finally, in a further embodiment, RNA aptamers can be introduced into or expressed in a cell. RNA aptamers are specific RNA ligands for proteins, such as for Tat and Rev RNA (see Good et al., Gene Ther., Vol. 4, pp. 45-54 (1997)) that can specifically inhibit their translation.

Methods of Modifying the Abundance or Activity of Expressed Protein

Methods of modifying protein abundance include, inter alia, those altering protein degradation rates and those using antibodies (which bind to proteins affecting abundance of activities of native target protein species). Methods of directly modifying protein activities include, inter alia, the use of antibodies, dominant negative mutations, specific drugs or chemical moieties.

Increasing (or decreasing) the degradation rates of a protein species decreases (or increases) the abundance of that species. Methods for increasing the degradation rate of a target protein in response to elevated temperature and/or exposure to a particular drug, which are known in the art, can be employed in this invention. For example, one such method employs a heat-inducible or drug-inducible N-terminal degron, which is an N-terminal protein fragment that exposes a degradation signal promoting rapid protein degradation at a higher temperature (e.g., 37° C.) and which is hidden to prevent rapid degradation at a lower temperature (e.g., 23° C.). See Dohmen et al., Science, Vol. 263, pp. 1273-1276 (1994). Such an exemplary degron is Arg-DHFR^{ts}, a variant of murine dihydrofolate reductase in which the N-terminal Val is replaced by Arg and the Pro at position 66 is replaced with Leu. According to this method, for example, a gene for a target protein, P, is replaced by standard gene targeting methods known in the art (see Lodish et al., Molecular Biology of the Cell, W.H. Freeman and Co., NY (1995), especially chap 8), with a gene coding for the fusion protein Ub-Arg-DHFR^{ts}-P (“Ub” stands for ubiquitin). The N-terminal ubiquitin is rapidly cleaved after translation exposing the N-terminal degron. At lower temperatures, lysines internal to Arg-DHFR^{ts} are not exposed, ubiquitination of the fusion protein does not occur, degradation is slow, and active target protein levels are high. At higher temperatures (in the absence of methotrexate), lysines internal to Arg-DHFR^{ts} are exposed, ubiquitination of the fusion protein occurs, degradation is rapid, and active target protein levels are low.

This technique also permits controllable modification of degradation rates since heat activation of degradation is controllably blocked by exposure methotrexate. This method is adaptable to other N-terminal degrons that are responsive to other inducing factors, such as drugs and temperature changes. Also, one of skill in the art will appreciate that expression of antibodies binding and inhibiting a target protein can be employed as another dominant negative strategy.

Modifying Expressed Protein Activity with Small Molecule Drugs or Ligands

In addition, the activities of certain target proteins can be modified or perturbed in a controlled or a saturating manner by exposure to exogenous drugs or ligands. Since the methods of this invention are often applied to testing or confirming the usefulness of various drugs to treat cancer, drug exposure is an important method of modifying/perturbing cellular constituents, both mRNAs and expressed proteins. In a preferred embodiment, input cellular constituents are perturbed either by drug exposure or genetic manipulation, such as gene deletion or knockout, and system responses are measured by gene expression technologies, such as hybridization to gene transcript arrays, described in the following.

In a preferable case, a drug is known that interacts with only one target protein in the cell and alters the activity of only that one target protein, either increasing or decreasing the activity. Graded exposure of a cell to varying amounts of that drug thereby causes graded perturbations of network models having that target protein as an input. Saturating exposure causes saturating modification/perturbation. For example, Cyclosporin A is a very specific regulator of the calcineurin protein, acting via a complex with cyclophilin. A titration series of Cyclosporin A therefore can be used to generate any desired amount of inhibition of the calcineurin protein. Alternately, saturating exposure to Cyclosporin A will maximally inhibit the calcineurin protein.

Modifying Protein Activity with Antibodies and Antagonists

The term “antagonist” refers to a molecule which, when bound to the protein encoded by the gene, inhibits its activity. Antagonists can include, but are not limited to, peptides, proteins, carbohydrates and small molecules.

In a particularly useful embodiment, the antagonist is an antibody specific for the cell-surface protein expressed by at least one gene. Antibodies useful as therapeutics encompass the antibodies, antibody derivatives, or antibody fragments as described above. The antibody alone may act as an effector of therapy or it may recruit other cells to actually effect cell killing. The antibody may also be conjugated to a reagent, such as a chemotherapeutic, radionuclide, ricin A chain, cholera toxin, pertussis toxin, etc., and serve as a target agent. Alternatively, the effector may be a lymphocyte carrying a surface molecule that interacts, either directly or indirectly, with a tumor target. Various effector cells include cytotoxic T-cells and NK-cells.

Examples of the antibody-therapeutic agent conjugates which can be used in therapy include, but are not limited to,
1) Antibodies coupled to radionuclides, such as ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho′, ¹⁷⁷Lu, ¹⁸⁶Re and ¹⁸⁶Re. See, e.g., in Goldenberg et al., Cancer Res., Vol. 41, pp. 4354-4360 (1981); Carrasquillo et al., Cancer Treat. Res., Vol. 68, pp. 317-328 (1984); Zalcberg et al.; J. Natl. Cancer Inst., Vol. 72, pp. 697-704 (1984); Jones et al., Int. J. Cancer, Vol. 35, pp. 715-720 (1985); Lange et al., Surgery, Vol. 98, pp. 143-150 (1985); Kaltovich et al., J. Nucl. Med., Vol. 27, pp. 897 (1986); Order et al., Int. J. Radiother. Oncol. Biol. Phys., Vol. 8, pp. 259-261 (1982); Courtenay-Luck et al., Lancet, Vol. 1, pp. 1441-1443 (1984); and Effinger et al., Cancer Treat. Res., Vol. 66, pp. 289-297 (1982);
2) Antibodies coupled to drugs or biological response modifiers, such as methotrexate, adriamycin and lymphokines, such as Interferon. See, e.g., Chabner et al., “Principles and Practice of Oncology”, Cancer, Vol. 1, pp. 290-328 (1985); Oldham et al., “Principles and Practice of Oncology”, Cancer, Vol. 2, pp. 2223-2245 (1985); Deguchi et al., Cancer Res., Vol. 46, pp. 43751-43755 (1986); Deguchi et al., Fed. Proc., Vol. 44, p. 1684 (1985); Embleton et al., Br. J. Cancer, Vol. 49, pp. 559-565 (1984); and Pimm et al., Cancer Immunol. Immunother., Vol. 12, pp. 125-134 (1982);
3) Antibodies coupled to toxins. See, e.g., Uhr et al., Monoclonal Antibodies and Cancer, Academic Press, Inc., pp. 85-98 (1983); Vitetta et al., Biotechnol. Bio. Frontiers, pp. 73-85 (1984); and Vitetta et al., Science, Vol. 219, pp. 644-650 (1983);
4) Heterofunctional antibodies, e.g., antibodies coupled or combined with another antibody so that the complex binds both to the carcinoma and effector cells, e.g., killer cells, such as T-cells. See, e.g., in Perez et al., J. Exper. Med., Vol. 163, pp. 166-178 (1986); and Lau et al., Proc. Natl. Acad. Sci. USA, Vol. 82, pp. 8648-8652 (1985); and
5) Native, i.e., non-conjugated or non-complexed, antibodies. See, e.g., Herlyn et al., Proc. Natl. Acad. Sci. USA, Vol. 79, pp. 4761-4765 (1982); Schulz et al., Proc. Natl. Acad. Sci. USA, Vol. 80, pp. 5407-5411 (1983); Capone et al., Proc. Natl. Acad. Sci. USA, Vol. 80, pp. 7328-7332 (1983); Sears et al., Cancer Res., Vol. 45, pp. 5910-5913 (1985); Nepom et al., Proc. Natl. Acad. Sci. USA, Vol. 81, pp. 2864-2867 (1984); Koprowski et al., Proc. Nat. Acad. Sci. USA, Vol. 81, pp. 216-219 (1984); and Houghton et al., Proc. Natl. Acad. Sci. USA, Vol. 82, pp. 1242-1246 (1985).

Methods for coupling an antibody, antibody derivatives, or antibody fragments to a therapeutic agent, as described above, are well-known in the art and are described, e.g., in the methods provided in the references above.

Use of an Antagonist as a Therapeutic

In yet another embodiment, the antagonist useful as a therapeutic for treating edema can be an inhibitor of a protein encoded by one of the disclosed genes.

Target protein activities can also be decreased by (neutralizing) antibodies. By providing for controlled or saturating exposure to such antibodies, protein abundance/activities can be modified or perturbed in a controlled or saturating manner. For example, antibodies to suitable epitopes on protein surfaces may decrease the abundance, and thereby indirectly decrease the activity, of the wild-type active form of a target protein by aggregating active forms into complexes with less or minimal activity as compared to the wild-type unaggregated wild-type form. Alternately, antibodies may directly decrease protein activity by, e.g., interacting directly with active sites or by blocking access of substrates to active sites. Conversely, in certain cases, (activating) antibodies may also interact with proteins and their active sites to increase resulting activity. In either case, antibodies (of the various types to be described) can be raised against specific protein species (by the methods to be described) and their effects screened. The effects of the antibodies can be assayed and suitable antibodies selected that raise or lower the target protein species concentration and/or activity. Such assays involve introducing antibodies into a cell (see below), and assaying the concentration of the wild-type amount or activities of the target protein by standard means (such as immunoassays) known in the art. The net activity of the wild-type form can be assayed by assay means appropriate to the known activity of the target protein.

Introduction of Antibodies into Cells

Antibodies can be introduced into cells in numerous fashions, including, for example, microinjection of antibodies into a cell (see Morgan et al., Immunol. Today, Vol. 9, pp. 84-86 (1988)) or transforming hybridoma mRNA encoding a desired antibody into a cell. See Burke et al., Cell, Vol. 36, pp. 847-858 (1984). In a further technique, recombinant antibodies can be engineering and ectopically expressed in a wide variety of non-lymphoid cell types to bind to target proteins as well as to block target protein activities. See Biocca et al., Trends Cell Biol., Vol. 5, pp. 248-252 (1995). Expression of the antibody is preferably under control of a controllable promoter, such as the Tet promoter, or a constitutively active promoter (for production of saturating perturbations). A first step is the selection of a particular monoclonal antibody with appropriate specificity to the target protein (see below). Then sequences encoding the variable regions of the selected antibody can be cloned into various engineered antibody formats, including, for example, whole antibody, Fab fragments, Fv fragments, single-chain Fv fragments (V_{H} and V_{L} regions united by a peptide linker) (“ScFv” fragments), diabodies (two associated ScFv fragments with different specificity), and so forth. See Hayden et al., Curr. Opin. Immunol., Vol. 9, pp. 210-212 (1997). Intracellularly expressed antibodies of the various formats can be targeted into cellular compartments (e.g., the cytoplasm, the nucleus, the mitochondria, etc.) by expressing them as fusion's with the various known intracellular leader sequences. See Bradbury et al., Antibody Engineering, Vol. 2, pp. 295-361 (1995). In particular, the ScFv format appears to be particularly suitable for cytoplasmic targeting.

The Variety of Useful Antibody Types

Antibody types include, but are not limited to, polyclonal, monoclonal, chimeric, single-chain, Fab fragments and an Fab expression library. Various procedures known in the art may be used for the production of polyclonal antibodies to a target protein. For production of the antibody, various host animals can be immunized by injection with the target protein, such host animals include, but are not limited to, rabbit, mice, rats, etc. Various adjuvants can be used to increase the immunological response, depending on the host species, and include, but are not limited to, Freunds (complete and incomplete), mineral gels, such as aluminum hydroxide, surface active substances such as lysolecithin, pluronic polyols, polyanions, peptides, oil emulsions, dinitrophenol, and potentially useful human adjuvants, such as Bacillus Calmette-Guerin (BCG) and corynebacterium parvum.

Monoclonal Antibodies

For preparation of monoclonal antibodies directed towards a target protein, any technique that provides for the production of antibody molecules by continuous cell lines in culture may be used. Such techniques include, but are not restricted to, the hybridoma technique originally developed by Kohler et al., Nature, Vol. 256, pp. 495-497 (1975), the trioma technique, the human B-cell hybridoma technique (see Kozbor et al., Immunol. Today, Vol. 4, p. 72 (1983)), and the EBV hybridoma technique to produce human monoclonal antibodies. See Cole et al., Monoclonal Antibodies Cancer Ther., pp. 77-96 (1985). In an additional embodiment of the invention, monoclonal antibodies can be produced in germ-free animals utilizing recent technology (PCT/US90/02545). According to the invention, human antibodies may be used and can be obtained by using human hybridomas (see Cote et al., Proc. Natl. Acad. Sci. USA, Vol. 80, pp. 2026-2030 (1983)), or by transforming human B cells with EBV virus in vitro. See Cole et al., (1985), supra. In fact, according to the invention, techniques developed for the production of “chimeric antibodies” (see Morrison et al. (1984), supra; Neuberger et al. (1984), supra; Takeda et al. (1985), supra, by splicing the genes from a mouse antibody molecule specific for the target protein together with genes from a human antibody molecule of appropriate biological activity can be used; such antibodies are within the scope of this invention.

Additionally, where monoclonal antibodies are advantageous, they can be alternatively selected from large antibody libraries using the techniques of phage display. See Marks et al., J. Biol. Chem., Vol. 267, pp. 16007-16010 (1992). Using this technique, libraries of up to 10¹² different antibodies have been expressed on the surface of fd filamentous phage, creating a “single pot” in vitro immune system of antibodies available for the selection of monoclonal antibodies. See Griffiths et al., EMBO J., Vol. 13, pp. 3245-3260 (1994). Selection of antibodies from such libraries can be done by techniques known in the art, including contacting the phage to immobilized target protein, selecting and cloning phage bound to the target, and subcloning the sequences encoding the antibody variable regions into an appropriate vector expressing a desired antibody format.

According to the invention, techniques described for the production of single-chain antibodies (see U.S. Pat. No. 4,946,778) can be adapted to produce single-chain antibodies specific to the target protein. An additional embodiment of the invention utilizes the techniques described for the construction of Fab expression libraries (see Huse et al. (1989), supra), to allow rapid and easy identification of monoclonal Fab fragments with the desired specificity for the target protein.

Antibody fragments that contain the idiotypes of the target protein can be generated by techniques known in the art. For example, such fragments include, but are not limited to, the F(ab′)₂ fragment which can be produced by pepsin digestion of the antibody molecule; the Fab′ fragments that can be generated by reducing the disulfide bridges of the F(ab′)₂ fragment, the Fab fragments that can be generated by treating the antibody molecule with papain and a reducing agent and Fv fragments.

In the production of antibodies, screening for the desired antibody can be accomplished by techniques known in the art, e.g., ELISA. To select antibodies specific to a target protein, one may assay generated hybridomas or a phage display antibody library for an antibody that binds to the target protein.

Other Methods of Modifying Protein Activities

Dominant negative mutations are mutations to endogenous genes or mutant exogenous genes that when expressed in a cell disrupt the activity of a targeted protein species. Depending on the structure and activity of the targeted protein, general rules exist that guide the selection of an appropriate strategy for constructing dominant negative mutations that disrupt activity of that target. See Hershkowitz, Nature, Vol. 329, pp. 219-222 (1987). In the case of active monomeric forms, over expression of an inactive form can cause competition for natural substrates or ligands sufficient to significantly reduce net activity of the target protein. Such over expression can be achieved by, for example, associating a promoter, preferably a controllable or inducible promoter, or also a constitutively expressed promoter, of increased activity with the mutant gene. Alternatively, changes to active site residues can be made so that a virtually irreversible association occurs with the target ligand. Such can be achieved with certain tyrosine kinases by careful replacement of active site serine residues. See Perlmutter et al., Curr. Opin. Immunol., Vol. 8, pp. 285-290 (1996).

In the case of active multimeric forms, several strategies can guide selection of a dominant negative mutant. Multimeric activity can be decreased in a controlled or saturating manner by expression of genes coding exogenous protein fragments that bind to multimeric association domains and prevent multimer formation. Alternatively, controllable or saturating over expression of an inactive protein unit of a particular type can tie up wild-type active units in inactive multimers, and thereby decrease multimeric activity. See Nocka et al., EMBO J., Vol. 9, pp. 1805-1813 (1990). For example, in the case of dimeric DNA binding proteins, the DNA binding domain can be deleted from the DNA binding unit, or the activation domain deleted from the activation unit. Also, in this case, the DNA binding domain unit can be expressed without the domain causing association with the activation unit. Thereby, DNA binding sites are tied up without any possible activation of expression. In the case where a particular type of unit normally undergoes a conformational change during activity, expression of a rigid unit can inactivate resultant complexes. For a further example, proteins involved in cellular mechanisms, such as cellular motility, the mitotic process, cellular architecture, and so forth, are typically composed of associations of many subunits of a few types. These structures are often highly sensitive to disruption by inclusion of a few monomeric units with structural defects. Such mutant monomers disrupt the relevant protein activities and can be expressed in a cell in a controlled or saturating manner.

In addition to dominant negative mutations, mutant target proteins that are sensitive to temperature (or other exogenous factors) can be found by mutagenesis and screening procedures that are well-known in the art.

Treatment Modalities

In the case of treatment with an antisense nucleotide, the method comprises administering a therapeutically effective amount of an isolated nucleic acid molecule comprising an antisense nucleotide sequence derived from the IL-1β gene, wherein the antisense nucleotide has the ability to change the transcription/translation of the IL-1β gene. The term “isolated” nucleic acid molecule means that the nucleic acid molecule is removed from its original environment, e.g., the natural environment if it is naturally-occurring. For example, a naturally-occurring nucleic acid molecule is not isolated, but the same nucleic acid molecule, separated from some or all of the co-existing materials in the natural system, is isolated, even if subsequently reintroduced into the natural system. Such nucleic acid molecules could be part of a vector or part of a composition and still be isolated, in that such vector or composition is not part of its natural environment.

With respect to treatment with a ribozyme or double-stranded RNA molecule, the method comprises administering a therapeutically effective amount of a nucleotide sequence encoding a ribozyme, or a double-stranded RNA molecule, wherein the nucleotide sequence encoding the ribozyme/double-stranded RNA molecule has the ability to change the transcription/translation of the IL-1β gene.

In the case of treatment with an antagonist, the method comprises administering to a subject a therapeutically effective amount of an antagonist that inhibits or activates a protein encoded by the IL-1β gene.

A “therapeutically effective amount” of an isolated nucleic acid molecule comprising an antisense nucleotide, nucleotide sequence encoding a ribozyme, double-stranded RNA, or antagonist, refers to a sufficient amount of one of these therapeutic agents to treat edema. The determination of a therapeutically effective amount is well within the capability of those skilled in the art. For any therapeutic, the therapeutically effective dose can be estimated initially in e.g. cell culture assays or in animal models, usually mice, rabbits, dogs or pigs. The animal model may also be used to determine the appropriate concentration range and route of administration. Such information can then be used to determine useful doses and routes for administration in humans.

Therapeutic efficacy and toxicity may be determined by standard pharmaceutical procedures in cell cultures or experimental animals, e.g., the dose therapeutically effective in 50% of the population (ED₅₀) and the dose lethal to 50% of the population (LD₅₀). The dose ratio between toxic and therapeutically effects is the therapeutic index, and it can be expressed as the ratio LD₅₀/ED₅₀. Antisense nucleotides, ribozymes, double-stranded RNAs and antagonists that exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range, depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to, maintain the desired effect. Factors that may be taken into account include the severity of the disease state, general health of the subject, age, weight and gender of the subject, diet, time and frequency of administration, drug combination(s), reaction sensitivities, and tolerance/response to therapy.

Normal dosage amounts may vary form 0.1-100,000 mg, up to a total dosage of about 1 g, depending upon the route of administration. Guidance as to particular dosages and methods of delivery is provided in the literature and generally available to practitioners in the art. Those skilled in the art will employ different formulations for nucleotides than for antagonists.

For therapeutic applications, the antisense nucleotides, nucleotide sequences encoding ribozymes, double-stranded RNAs (whether entrapped in a liposome or contained in a viral vector) and antibodies are preferably administered as pharmaceutical compositions containing the therapeutic agent in combination with one or more pharmaceutically acceptable carriers. The compositions may be administered alone or in combination with at least one other agent, such as stabilizing compound, which may be administered in any sterile, biocompatible pharmaceutical carrier, including, but not limited to, saline, buffered saline, dextrose and water. The compositions may be administered to a patient alone or in combination with other agents, drugs or hormones.

The pharmaceutical compositions may be administered by an number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual or rectal means. In addition to the active ingredient, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's “Pharmaceutical Sciences”, Maack Publishing Co., Easton, Pa.

Pharmaceutical compositions for oral administration can be formulated using pharmaceutically acceptable carriers well-known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients re carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins, such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations, which can be used orally, include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycatonic amino polymers may also be used for delivery. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with many acids including, but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder that may contain any or all of the following: 1-50 mM histidine, 0.1-2% sucrose and 2-7% mannitol, at a pH range of 4.5-5.5, that is combined with buffer prior to use.

After pharmaceutical compositions have been prepared, they can be placed in an appropriate container and labeled for treatment of an indicated condition. For administration of the antisense nucleotide or antagonist, such labeling would include amount, frequency, and method of administration. Those skilled in the art will employ different formulations for antisense nucleotides than for antagonists, e.g., antibodies or inhibitors. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

In another aspect, a method for treating edema in a subject is provided which utilizes a therapeutic agent as described above, e.g., an antisense nucleotide, a ribozyme, a double-stranded RNA, and an antagonist, such as an antibody. With respect to treating edema utilizing an antisense nucleotide, the method comprises administering to the subject a therapeutically effective amount of an isolated nucleic acid molecule comprising an antisense nucleotide sequence derived from the IL-1β gene, wherein the antisense nucleotide has the ability to change the transcription/translation of the at least one gene.

With respect to the treatment of edema utilizing a ribozyme, such a method comprises administering to the subject a therapeutically effective amount of a nucleotide sequence encoding the ribozyme, which has the ability to change the transcription/translation of the IL-1β gene.

With respect to treatment of edema utilizing a double-stranded RNA, the method comprises administering to the subject a therapeutically effective amount of a double-stranded RNA corresponding to the IL-1β gene, wherein the double-stranded RNA has the ability to change the transcription/translation of the IL-1β gene.

With respect to treatment of edema utilizing an antagonist, the method comprises administering to the subject a therapeutically effective amount of an antagonist that results in inhibition or activation of a protein encoded by the IL-1β gene.

In the context of treating edema, a “therapeutically effective amount” of an isolated nucleic acid molecule comprising an antisense nucleotide, a nucleotide sequence encoding a ribozyme, a double-stranded RNA, or antagonist, refers to a sufficient amount of one of these therapeutic agents to reduce the degree of edema and can be determined as described above.

Computer Implementations

In a preferred embodiment, the computation steps of the previous methods are implemented on a computer system or on one or more networked computer systems in order to provide a powerful and convenient facility for forming and testing models of biological systems. The computer system may be a single hardware platform comprising internal components and being linked to external components. The internal components of this computer system include processor element interconnected with a main memory. For example computer system can be an Intel Pentium based processor of 200 Mhz or greater clock rate and with 32 MB or more of main memory.

The external components include mass data storage. This mass storage can be one or more hard disks (which are typically packaged together with the processor and memory). Typically, such hard disks provide for at least 1 GB of storage. Other external components include user interface device, which can be a monitor and keyboards, together with pointing device, which can be a “mouse”, or other graphic input devices. Typically, the computer system is also linked to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on mass storage. Alternatively, the software components may be stored on removable media such as floppy disks or CD-ROM (not illustrated). The software component represents the operating system, which is responsible for managing the computer system and its network interconnections. This operating system can be, e.g., of the Microsoft Windows family, such as Windows 95, Windows 98 or Windows NT, or a Unix operating system, such as Sun Solaris. Software includes common languages and functions conveniently present on this system to assist programs implementing the methods specific to this invention. Languages that can be used to program the analytic methods of this invention include C, C++, or, less preferably, JAVA. Most preferably, the methods of this invention are programmed in mathematical software packages, which allow symbolic entry of equations and high-level specification of processing, including algorithms to be used, and thereby freeing a user of the need to procedurally program individual equations or algorithms. Such packages include, e.g., MATLAB™ from Mathworks (Natick, Mass.), MATHEMATICA™ from Wolfram Research (Champaign, Ill.) and MATHCAD™ from Mathsoft (Cambridge, Mass.).

In preferred embodiments, the analytic software component actually comprises separate software components that interact with each other. Analytic software represents a database containing all data necessary for the operation of the system. Such data will generally include, but is not necessarily limited to, results of prior experiments, genome data, experimental procedures and cost, and other information, which will be apparent to those skilled in the art. Analytic software includes a data reduction and computation component comprising one or more programs which execute the analytic methods of the invention. Analytic software also includes a user interface which provides a user of the computer system with control and input of test network models, and, optionally, experimental data. The user interface may comprise a drag-and-drop interface for specifying hypotheses to the system. The user interface may also comprise means for loading experimental data from the mass storage component (e.g., the hard drive), from removable media (e.g., floppy disks or CD-ROM), or from a different computer system communicating with the instant system over a network (e.g., a local area network, or a wide area communication network, such as the Internet).

Alternative computer systems and methods for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

### Glossary

Allele A particular form of a gene or DNA sequence at a specific chromosomal location (locus).
Antibodies Includes polyclonal and monoclonal antibodies, chimeric, single-chain, and humanized antibodies, as well as Fab fragments, including the products of an Fab or other immunoglobulin expression library.
Candidate gene A gene which is hypothesized to be responsible for a disease, condition, or the response to a treatment, or to be correlated with one of these.
Full-geneotype The unphased 5′ to 3′ sequence of nucleotide pairs found at all known polymorphic sites in a locus on a pair of homologous chromosomes in a single individual.
Full-haplotype The 5′ to 3′ sequence of nucleotides found at all known polymorphic sites in a locus on a single chromosome from a single individual.
Gene A segment of DNA that contains all the information for the regulated biosynthesis of an RNA product, including promoters, exons, introns, and other untranslated regions that control expression.
Genotype An unphased 5′ to 3′ sequence of nucleotide pair(s) found at one or more polymorphic sites in a locus on a pair of homologous chromosomes in an individual. As used herein, genotype includes a full-genotype and/or a sub-genotype as described below.
Genotyping A process for determining a genotype of an individual.
Haplotype A 5′ to 3′ sequence of nucleotides found at one or more linked polymorphic sites in a locus on a single chromosome from a single individual.
Haplotype data Information concerning one or more of the following for a specific gene: a listing of the haplotype pairs in each individual in a population; a listing of the different haplotypes in a population; frequency of each haplotype in that or other populations, and any known associations between one or more haplotypes and a trait.
Haplotype pair Two haplotypes found for a locus in a single individual.
Haplotyping A process for determining one or more haplotypes in an individual and includes use of family pedigrees, molecular techniques and/or statistical inference.
Homolog A generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog” and “paralog”.
Identity A relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.
Isoform A particular form of a gene, mRNA, cDNA or the protein encoded thereby, distinguished from other forms by its particular sequence and/or structure.
Isogene One of the isoforms of a gene found in a population. An isogene contains all of the polymorphisms present in the particular isoform of the gene.
Isolated As applied to a biological molecule, such as RNA, DNA, oligonucleotide or protein; isolated means the molecule is substantially free of other biological molecules, such as nucleic acids, proteins, lipids, carbohydrates, or other material, such as cellular debris and growth media. Generally, the term “isolated” is not intended to refer to a complete absence of such material or to absence of water, buffers, or salts, unless they are present in amounts that substantially interfere with the methods of the present invention.
Linkage Describes the tendency of genes to be inherited together as a result of their location on the same chromosome; measured by percent recombination between loci.
Linkage disequilibrium Describes a situation in which some combinations of genetic markers occur more or less frequently in the population than would be expected from their distance apart. It implies that a group of markers has been inherited coordinately. It can result from reduced recombination in the region or from a founder effect, in which there has been insufficient time to reach equilibrium since one of the markers was introduced into the population.
Locus A location on a chromosome or DNA molecule corresponding to a gene or a physical or phenotypic feature.
Modified bases Include, e.g., tritylated bases and unusual bases, such as inosine. A variety of modifications may be made to DNA and RNA; thus, polynucleotide embraces chemically, enzymatically or metabolically modified forms of polynucleotides as typically found in nature, as well as the chemical forms of DNA and RNA characteristic of viruses and cells. Polynucleotide also embraces relatively short polynucleotides, often referred to as oligonucleotides.
Naturally-occurring A term used to designate that the object it is applied to, e.g., naturally-occurring polynucleotide or polypeptide, can be isolated from a source in nature and which has not been intentionally modified by man.
Nucleotide pair The nucleotides found at a polymorphic site on the two copies of a chromosome from an individual.
Ortholog A polynucleotide or polypeptide that is the functional equivalent of the polynucleotide or polypeptide in another species.
Paralog A polynucleotide or polypeptide that within the same species which is functionally similar.
Phased As applied to a sequence of nucleotide pairs for two or more polymorphic sites in a locus, phased means the combination of nucleotides present at those polymorphic sites on a single copy of the locus is known.
Polymorphic site (PS) A position within a locus at which at least two alternative sequences are found in a population, the most frequent of which has a frequency of no more than 99%.
Polymorphic variant A gene, mRNA, cDNA, polypeptide or peptide whose nucleotide or amino acid sequence varies from a reference sequence due to the presence of a polymorphism in the gene.
Polymorphism Any sequence variant present at a frequency of >1% in a population. The sequence variation observed in an individual at a polymorphic site. Polymorphisms include nucleotide substitutions, insertions, deletions and microsatellites and may, but need not, result in detectable differences in gene expression or protein function.
Polymorphism data Information concerning one or more of the following for a specific gene: location of polymorphic sites; sequence variation at those sites; frequency of polymorphisms in one or more populations; the different genotypes and/or haplotypes determined for the gene; frequency of one or more of these genotypes and/or haplotypes in one or more populations; any known association(s) between a trait and a genotype or a haplotype for the gene.
Polymorphism database A collection of polymorphism data arranged in a systematic or methodical way and capable of being individually accessed by electronic or other means.
Polynucleotide Any RNA or DNA, which may be unmodified or modified RNA or DNA. Polynucleotides include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.
Polypeptide Any polypeptide comprising two or more amino acids joined to each other by peptide bonds or modified peptide bonds, i.e., peptide isosteres. Polypeptide refers to both short chains, commonly referred to as peptides, oligopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.
Population group A group of individuals sharing a common characteristic, such as ethnogeographic origin, medical condition, response to treatment etc.
Reference population A group of subjects or individuals who are predicted to be representative of one or more characteristics of the population group. Typically, the reference population represents the genetic variation in the population at a certainty level of at least 85%, preferably at least 90%, more preferably at least 95% and even more preferably at least 99%.
Single Nucleotide Polymorphism (SNP) The occurrence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bp from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.
Splice variant cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of which may encode different amino acid sequences. The term “splice variant” also refers to the proteins encoded by the above cDNA molecules.
Sub-genotype The unphased 5′ to 3′ sequence of nucleotides seen at a subset of the known polymorphic sites in a locus on a pair of homologous chromosomes in a single individual.
Sub-haplotype The 5′ to 3′ sequence of nucleotides seen at a subset of the known polymorphic sites in a locus on a single chromosome from a single individual.
Subject A human individual whose genotypes or haplotypes or response to treatment or disease state are to be determined.
Treatment A stimulus administered internally or externally to a subject.
Unphased As applied to a sequence of nucleotide pairs for two or more polymorphic sites in a locus, unphased means the combination of nucleotides present at those polymorphic sites on a single copy of the locus is not known.
See also, Human Molecular Genetics, 2^{nd} edition. Tom Strachan and Andrew P. Read. John Wiley and Sons, Inc. Publication, New York,
   References Cited

All publications and references, including but not limited to publications, patents, patent applications, GenBank accession, Unigene Cluster numbers and protein accession numbers, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

1. A method to predict which patient will be likely to develop edema when treated with a drug comprising the steps of:
a) determining RNA expression levels in a biological sample for a plurality of the 13 predictor genes shown in Table 2;
b) comparing patients gene expression profile to the mean No Edema expression profiles shown in Table 3;
c) determining the similarity between the two gene expression profiles resulting from the comparison in (b);
d) determining the likelihood that the patient will develop edema when treated with a drug by means of the degree of similarity determined in (c).

2. The method of claim 1, wherein the similarity determined in (c) is the mathematical correlation coefficient obtained by comparing the said two gene expression profiles.

3. The method of claim 2, wherein the correlation coefficient determined in (c) is the Pearson Correlation Coefficient (PCC).

4. The method of claim 3, wherein step (d) comprises determining that the patient will be more likely to develop edema than not, when treated with a drug, if the PCC is <0.37; and determining that the patient will be more likely not to develop edema than to develop it if the PCC is ≧0.37.

5. A method to predict, with high sensitivity, which patients will be more likely to develop edema when treated with a drug, such that no more than 15% of Edema cases will be misclassified as having No Edema, comprising the steps of:
a) determining RNA expression levels in a biological sample for a plurality of the 13 predictor genes shown in Table 2;
b) comparing patients gene expression profile to the mean No Edema expression profiles shown in Table 3;
c) determining the PCC between the two gene expression profiles resulting from the comparison in (b);
d) determining that the patient will be more likely to develop edema than not, when treated with a drug, if the PCC is negative and <0.78; and
e) determining that the patient will be more likely not to develop edema than to develop it if the negative PCC is ≧0.78.

6. The method of claim 1, wherein the biological sample comprises a blood sample.

7. The method of claim 1, wherein all the 13 predictor genes in Table 2 are used.

8. The method of claim 1, wherein the drug is a tyrosine kinase inhibitor (TKI).

9. The method of claim 8, wherein the TKI is Imatinib or GLEEVEC™/GLIVEC®).

10. A method to predict which female patient will be likely to develop edema when treated with a drug, comprising the steps of:
a) determining for the two copies of the IL-1β gene, present in the patient, the identity of the nucleotide pairs at the polymorphic site at position −511 base pairs upstream (at position 1423 of sequence X04500) from the transcriptional start site; and
b) determining that the patient will be likely to develop edema if both nucleotide pairs at this site are GC and determining that the patient will not be likely to develop edema if at least one nucleotide pair at this site is AT.

11. The method of claim 10, wherein the drug is a TKI.

12. The method of claim 11, wherein the TKI is Imatinib or GLEEVEC™/GLIVEC®.

13. A method to predict which female patient will be likely to develop edema when treated with a drug, comprising the steps of:
a) determining for the two copies of the IL-1β gene, present in the patient, the identity of the nucleotide pairs at the polymorphic site at position −31 base pairs upstream (at position 1903 of sequence X04500) from the transcriptional start site; and
b) determining that the patient will be likely to develop edema if both nucleotide pairs at this site are AT and determining that the patient will not be likely to develop edema if at least one nucleotide pair at this site is GC.

14. The method of claim 13, wherein the drug is a TKI.

15. The method of claim 14, wherein the TKI is Imatinib or GLEEVEC™/GLIVEC®.

16. A method to predict which female patient will be likely to develop edema when treated with a drug, comprising the steps of:
a) determination of the level of transcription of the IL-1β gene in a biological sample; and
b) determining that the patient would be likely to develop edema when treated with a drug if the level is above a threshold level.

17. A method to predict which female patient will be more likely to develop edema when treated with a drug, comprising the steps of:
a) determination of the level of the protein expressed by the IL-1β gene in a biological sample; and
b) determining that the patient would be likely to develop edema when treated with a drug if the level is above a threshold level.

18. The method of claim 16, wherein the drug is a TKI.

19. The method of claim 18, wherein the TKI is Imatinib or GLEEVEC™/GLIVEC®.

20. A method to predict which patient will be likely to develop edema when treated with a drug comprising the steps of:
a) determining the pattern of protein expression in a biological sample for two or more of the protein products of the 13 predictor genes shown in Table 2;
b) comparing the pattern of protein expression with the pattern expected for the Edema and the No Edema expression profile shown in Table 3;
c) determining that if the pattern is more similar to the No Edema pattern that the patient will not be likely to develop edema when treated with a drug; and
d) determining that if the pattern is more similar to the Edema pattern that the patient will be likely to develop edema when treated with a drug.

21. The method of claim 20, wherein the protein expression of a plurality of the 13 predictor genes shown in Table 2 is determined.

22. The method of claim 21, wherein the protein expression of all the 13 predictor genes shown in Table 2 is determined.

23. The method of claim 20, wherein the drug is a TKI.

24. The method of claim 23, wherein the TKI is Imatinib or GLEEVEC™/GLIVEC®.

25. (canceled)

26. A method to design clinical trials for the testing of drugs comprising the steps of:
a) determining by the use of either expression profiling or genotyping methods the likelihood that a particular patient will develop edema when exposed to the test drug; and
b) assigning that patient to the appropriate classification in the clinical trial based on the results of the determination in (a).

27-42. (canceled)

43. A kit for predicting which patient will be likely to develop edema when treated with a drug comprising:
(a) a means for determining the pattern of protein expression corresponding to the two or more of the 13 predictor genes shown in Table 2;
(b) a container suitable for containing the means and the biological sample of the patient comprising the proteins, wherein the means can form complexes with the proteins;
(c) a means to detect the complexes of (b); and
(d) instructions for use and interpretation of the kit results.

44. (canceled)

45. A kit for predicting which patient will be likely to develop edema when treated with a drug comprising:
(a) a means for determining the level of the protein expressed by the IL-1β gene;
(b) a container suitable for containing the means and the biological sample of the patient comprising the protein, wherein the means can form complexes with the protein;
(c) a means to detect the complexes of (b); and
(d) instructions for use and interpretation of the kit results.

46. The method of claim 17, wherein the determination step (a) further comprises the use of a kit of claim 37.

47. The method of claim 20, wherein the determination step (a) further comprises the use of a kit of claim 34.

48-71. (canceled)

72. A kit for determining the identity of the nucleotide pair at the −511 position of the IL-1β gene (at position 1423 of sequence X04500) from the transcriptional start site for the two copies of the IL-1β gene present in the patient; comprising:
a) a container comprising at least one reagent specific for detecting the nature of the nucleotide pair at the at the −511 position of the IL-1β gene (at position 1423 of sequence X04500) from the transcriptional start site for the two copies of the IL-1β gene present in the patient; and
b) instructions for interpreting the results based on the nature of the said nucleotide pair.

73. A kit for determining the identity of the nucleotide pair at the polymorphic site at position −31 base pairs upstream (at position 1903 of sequence X04500) from the transcriptional start site; comprising:
a) a container comprising at least one reagent specific for detecting the nature of the nucleotide pairs at the polymorphic site at position −31 base pairs upstream (at position 1903 of sequence X04500) from the transcriptional start site; and
b) instructions for interpreting the results based on the nature of the said nucleotide pair.

74-75. (canceled)
